(19) United States
(12) Patent Application Publication
(10) Pub. No.: US 2024/0238444 A1
(43) Pub. Date: Jul. 18, 2024

(54) COMPOSITION FOR PREVENTING OR TREATING PROGERIA AND NATURAL AGING THROUGH GENE EDITING

(71) Applicant: KOREA RESEARCH INSTITUTE OF BIOSCIENCE AND BIOTECHNOLOGY, Daejeon (KR)

(72) Inventors: Sun Uk KIM, Daejeon (KR); Young Ho PARK, Daejeon (KR); Seung Hwan LEE, Daejeon (KR); Jong Hee LEE, Daejeon (KR); Jae Jin CHA, Daejeon (KR); Han Seop KIM, Daejeon (KR); Un Bin CHAE, Daejeon (KR)

(73) Assignee: KOREA RESEARCH INSTITUTE OF BIOSCIENCE AND BIOTECHNOLOGY, Daejeon (KR)

(21) Appl. No.: 18/310,969

(22) Filed: May 2, 2023

Related U.S. Application Data
(63) Continuation-in-part of application No. PCT/KR2021/015531, filed on Nov. 1, 2021.

(30) Foreign Application Priority Data
Nov. 3, 2020 (KR) 10-2020-0145160
Oct. 28, 2021 (KR) 10-2021-0145418

(51) Int. Cl. A61K 48/00; A61K 31/7105; A61K 38/46; A61P 39/00; C12N 9/22; C12N 15/11

(52) U.S. Cl. CPC A61K 48/0033; A61K 31/7105; A61K 38/465; A61P 39/00; C12N 9/22; C12N 15/11; C12N 2310/20

(57) ABSTRACT

Provided is a composition for the prevention or treatment of Hutchinson-Gilford Progeria syndrome (HGPS) using gene editing, which contains sgRNA that hybridizes to mRNA encoding progerin, which causes HGPS, and a gene encoding Cas13 protein acting on the same. When introduced into the cell of a subject to be treated and only the mRNA encoding progerin is selectively cut. There is no need for co-prescribing with other therapies and fewer side effects occur than traditional farnesyltransferase inhibitors (FTIs). The efficiency is higher than when treated using homologous recombination (HR) at the DNA level, treatment using composition can be made reversibly, and the composition can be applied specifically compared to targeted treatment using RNAi (RNA interference), and has fewer side effects. Compared to treatment using CRISPR/Cas9, which directly acts on DNA and produces irreversible results, treatment using composition is reversible and selectively cut only mRNA encoding progerin, thereby ensuring safety.

### CROSS REFERENCE TO RELATED APPLICATIONS

This application is a Continuation of PCT International Application No. PCT/KR2021/015531 filed Nov. 1, 2021, claiming priorities based on Korean Patent Application No. 10-2020-0145160 filed Nov. 3, 2020 and Korean Patent Application No. 10-2021-0145418 filed Oct. 28, 2021.

### INCORPORATION BY REFERENCE OF SEQUENCE LISTING

The content of the electronically submitted sequence listing, file name: Q287293_Sequence listing.XML; size: 152,242 bytes; and date of creation: May 2, 2023, filed herewith, is incorporated herein by reference in its entirety.

### TECHNICAL FIELD

The present disclosure relates to a composition for preventing or treating Hutchinson-Gilford Progeria syndrome (HGPS) and natural aging using gene editing.

### BACKGROUND ART

Hutchinson-Gilford Progeria syndrome (HGPS) is a rare genetic disease caused by a point mutation (p.G608G, c.C1824T) on the lamin A (LMNA) locus. Among the patients investigated to date, a high proportion of patients have point mutations (p.G608G, c.C1824T) in the LMNA gene. Unlike normal gene expression, these genetic mutations induce alternative splicing, resulting in the expression of an abnormal lamin A protein (progerin), which accumulates in the nuclear membrane and interferes with the function of normal lamin A, leading to cell death. Studies have also reported that progerin increases with age and exposure to ultraviolet (UV) light in normal people, which may contribute to aging [2014, Aging, Pacheco et al., 2013, J Invest Dermatol, Takeuchi et al., 2011, J Clin Invest, Cao et al.]. The resulting phenotype is named HGPS, and various methods for treating the same have been attempted.

Recently, attempts have been made to target modified proteins at the genetic level to reduce side effects or resistance to therapeutics. Representative attempts have been made using RNAi (RNA interference) or HR (Homologous recombination) [2005, Hum Genet, Shurong Huang et al., 2011, Cell Stem Cell, Guang-Hui Liu et al.]. However, existing methods using RNAi or HR suffer from low efficiency or inaccurate targeting of specific genes. With the recent development of CRISPR/Cas9 technology, studies on progerin treatment have been reported [2019, Nat Med, Ergin Beyret et al., 2019 Nat Med, OlayaSantiago-Fernandez et al.]. However, because CRISPR/Cas9 cannot distinguish between lamin A and progerin on DNA, it was unable to cleave only progerin, which is responsible for HGPS disease, and normal lamin A was also reduced. The biggest drawback of the CRISPR/Cas9 system is that it also binds to and cleave off-targets. Accordingly, once the DNA has been cleaved, repairing the same into the original thereof is impossible, thereby causing stability issues.

To address this issue, an RNA gene targeting system (CRISPR/Cas13) was introduced to overcome this irreversible limitation of CRISPR/Cas9 and select for progerin (specifically, progerin mRNA), which is precisely what causes the problem in HGPS. The RNA gene targeting system (CRISPR/Cas13) is a type of bacterial immune system that operates based on a protein called Cas13 and a guide RNA complementary to target RNA, and has the ability to precisely cut the target RNA to regulate its expression. The present disclosure is designed to enable non-permanent and effective treatment by targeting progeria-inducing genetic mutations at the RNA level based on a target-specific RNA gene targeting system (CRISPR/Cas13).

### DETAILED DESCRIPTION OF THE DISCLOSURE

### Technical Problem

An aspect of the present disclosure is to provide a polynucleotide encoding a gRNA that hybridizes to progerin mRNA, wherein the polynucleotide is one of SEQ ID NOs: 1 to 5.

Another aspect of the present disclosure is to provide a gRNA derived from a polynucleotide of one of SEQ ID NOs: 1 to 5.

Another aspect of the present disclosure is to provide a composition for inhibiting the expression of progerin including gRNA derived from a polynucleotide of one of SEQ ID NOs: 1 to 5, and Cas13 mRNA or a Cas13 protein derived therefrom.

Another aspect of the present disclosure is to provide a recombinant expression vector including a polynucleotide sequence of one of SEQ ID NOs: 1 to 5 encoding a gRNA that hybridizes to progerin mRNA or a sequence complementary thereto.

Another aspect of the present disclosure is to provide a composition for inhibiting the expression of progerin containing the recombinant expression vector as an active ingredient or a composition for inhibiting the expression of progerin containing the recombinant expression vector and the Cas13 expression vector.

Another aspect of the present disclosure is to provide a pharmaceutical composition for preventing or treating Hutchinson-Gilford Progeria syndrome (HGPS) and natural aging including the composition as an active ingredient.

Another aspect of the present disclosure is to provide a method of alleviating or treating HGPS and natural aging including administering the composition to a subject in need thereof.

Another aspect of the present disclosure is to provide use of the composition for alleviating or treating HGPS and natural aging.

Another aspect of the present disclosure is to provide use of the composition for producing a pharmaceutical composition for alleviating or treating HGPS and natural aging.

### Technical Solution to Problem

The present disclosure provides a polynucleotide encoding a gRNA that hybridizes to progerin mRNA, wherein the polynucleotide is one of SEQ ID NOs: 1 to 5.

In addition, the present disclosure provides a gRNA derived from the polynucleotide of one of SEQ ID NOs: 1 to 5.

In an embodiment of the present disclosure, the gRNA may hybridize to genes encoding exon 11 and exon 12 of LMNA gene.

The present disclosure provides a composition for inhibiting the expression of progerin including gRNA derived from a polynucleotide of one of SEQ ID NOs: 1 to 5, and Cas13 mRNA or a Cas13 protein derived therefrom.

The present disclosure provides a recombinant expression vector including a polynucleotide sequence of one of SEQ ID NOs: 1 to 5 encoding a gRNA that hybridizes to progerin mRNA or a sequence complementary thereto.

In an embodiment of the present disclosure, the recombinant expression vector may further include a polynucleotide sequence encoding Cas13.

In addition, in an embodiment of the present disclosure, the recombinant expression vector may consist of any one sequence of SEQ ID NOs: 6 to 15.

The present disclosure provides a composition for inhibiting the expression of progerin including the recombinant expression vector as an active ingredient.

The present disclosure provides a composition for inhibiting the expression of progerin including the recombinant expression vector and the Cas13 expression vector.

The present disclosure provides a pharmaceutical composition for preventing or treating HGPS and natural aging including the composition as an active ingredient.

The present disclosure provides a method of alleviating or treating HGPS and natural aging including administering the composition to a subject in need thereof.

The present disclosure provides use of the composition for alleviating or treating HGPS and natural aging.

The present disclosure provides use of the composition for producing a pharmaceutical composition for alleviating or treating HGPS and natural aging.

### Advantageous Effects of Disclosure

The recombinant expression vector of the present disclosure and the pharmaceutical composition for preventing or treating Hutchinson-Gilford Progeria syndrome (HGPS) and natural aging including the same, each include sgRNA that hybridizes to mRNA encoding progerin, which causes HGPS, and a gene encoding Cas13 protein acting on the sgRNA. Accordingly, the recombinant expression vector is introduced into the cells of a subject in need thereof and only the mRNA encoding progerin is optionally cleaved. When the composition is used, there is no need for co-prescribing with other therapies and fewer side effects occur than traditional farnesyltransferase inhibitors (FTIs). In addition, the efficiency is higher than when treated using homologous recombination (HR) at the DNA level, treatment using the composition can be made reversibly, and the composition can be applied specifically compared to targeted treatment using RNAi (RNA interference), and has fewer side effects. In addition, compared to treatment using CRISPR/Cas9, which directly acts on DNA and produces irreversible results, treatment using the composition is reversible and can selectively cut only mRNA encoding progerin, thereby ensuring safety.

### BRIEF DESCRIPTION OF DRAWINGS

FIG. 1 is a diagram showing a mutation that causes Hutchinson-Gilford Progeria syndrome (HGPS).

FIG. 2 is a diagram showing the mechanism of action of Cas13 used in the present disclosure.

FIG. 3 is a view showing positions in progerin mRNA to which sgRNAs encoded by SEQ ID NOs: 1 to 5 of present disclosure hybridize.

FIGS. 4 to 9 are views showing the vector used as the recombinant expression vector of the present disclosure in Example 1 (FIG. 4) and recombinant expression vectors of the present disclosure (FIGS. 5 to 9).

FIGS. 10 and 11 show a lamin A overexpression vector (FIG. 10) and a progerin overexpression vector (FIG. 11) used in Experimental Example 2-1.

FIGS. 12A to 12C shows the vectors used in LMNA conditionally overexpressing cell lines and Progerin conditionally overexpressing cell lines in Experimental Example 2-2, in which, for the construction of LMNA conditionally overexpressing cell lines, AAVS1 all-in one, H11 all-in one, ERT2CreERT2, and Conditional LMNA vectors were used, and for the construction of Progerin conditionally overexpressing cell lines, AAVS1 all-in one, H11 all-in one, ERT2CreERT2, and Conditional Progerin vectors were used.

FIGS. 13A and 13B shows a schematic diagram of cell line verification insertion of LMNA conditionally overexpressing cell lines and Progerin conditionally overexpressing cell lines in Experimental Example 2-2.

FIGS. 14A to 14E shows a result of confirming whether a nucleotide sequence is inserted into LMNA conditionally overexpressing cell lines and Progerin conditionally overexpressing cell lines according to Experimental Example 2-2, wherein the WT band is a cell line in which the exogenous overexpression vector is not inserted, and the two bands, WT and Tg, show that only one allele has the gene inserted (heterogeneous).

FIGS. 15A to 15C shows the result of verifying whether the vector, inserted according to Experimental Example 2-2, conditionally expresses a gene, through fluorescence or a RT-PCR method.

FIGS. 16A to 16D is a diagram showing the location of LMNA semi RT-PCR primer on the gene in Experimental Example 2-2.

FIGS. 17A to 17D is a diagram showing the location of Progerin semi RT-PCR primer on the gene in Experimental Example 2-2.

FIGS. 18A and 18B is a diagram showing the location of LMNA specific RT-PCR primer on the gene in Experimental Example 2-3.

FIGS. 19A to 19C is a diagram showing the location of Progerin specific RT-PCR primer on the gene in Experimental Example 2-3.

FIGS. 20 and 21 show in vitro results of Experimental Example 2-3, from which it was confirmed that the recombinant expression vector of the present disclosure resulted in the reduction in progerin mRNA and progerin protein.

FIGS. 22 and 23 show results of Experimental Example 3, from which it was confirmed that the recombinant expression vector of the present disclosure resulted in the reduction in progerin mRNA and progerin protein.

FIG. 24 shows a result of Experimental Example 4-1, including the fluorescence analysis results of the experimental group into which the recombinant expression vector of the present disclosure was introduced.

FIGS. 25 and 26 show the results of Experimental Example 4-2, from which it can be confirmed that progerin and the expression of aging related genes were reduced in the experimental group into which the recombinant expression vector of the present disclosure was introduced.

FIG. 27 is a view showing positions in progerin mRNA to which sgRNAs encoded by SEQ ID NOs: 1 to 5 of present disclosure hybridize.

FIGS. 28 to 33 are views showing the vector used as the recombinant expression vector of the present disclosure in Example 2 (FIG. 28) and recombinant expression vectors of the present disclosure (FIGS. 29 to 33).

FIGS. 34 to 37 show in vitro results of Experimental Examples 5-1 and 5-2, from which it was confirmed that the recombinant expression vector of the present disclosure resulted in the reduction in progerin mRNA and progerin protein.

FIGS. 38 to 41 show the results of Experimental Examples 6-1 to 6-3 using fibroblasts of the HGPS mouse model, from which it can be confirmed that the recombinant expression vector of the present disclosure has the alleviation effects on the HGPS phenotype and HGPS-induced aging and active oxygen.

FIG. 42 shows the results of Experimental Example 6-4 using fibroblasts derived from a patient with HGPS, from which it can be confirmed that the recombinant expression vector of the present disclosure had the progerin mRNA reduction effect.

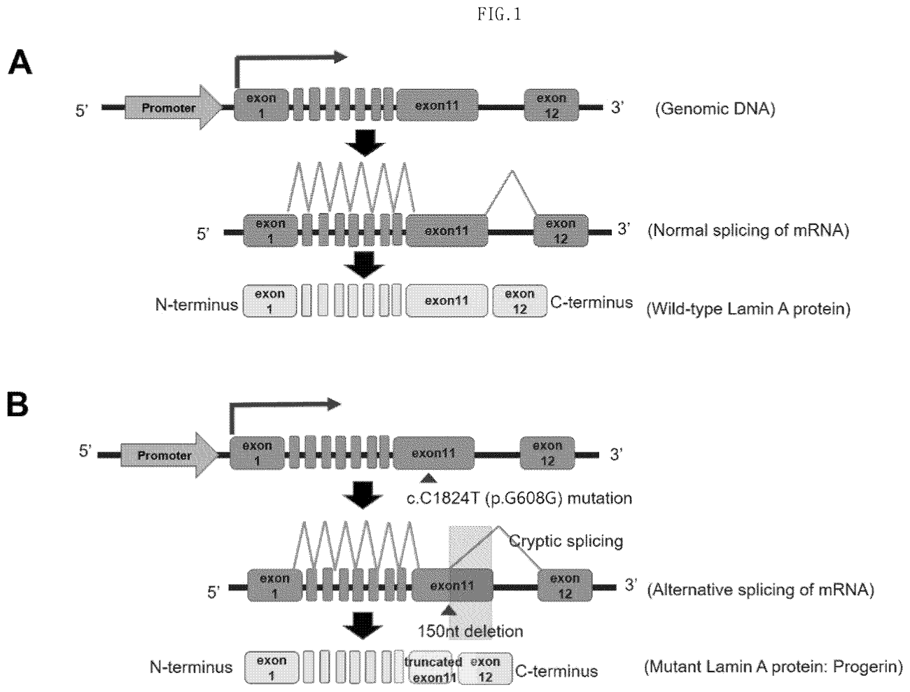

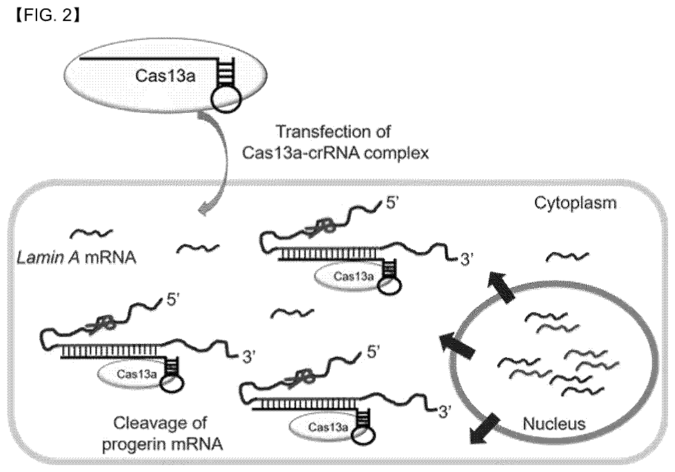

### BEST MODE

An aspect of the present disclosure is to provide a polynucleotide encoding a gRNA that hybridizes to progerin mRNA, wherein the polynucleotide is one of SEQ ID NOs: 1 to 5.

The term “progerin” used herein refers to an abnormal expression form of the lamin A protein, specifically an abnormal lamin A protein expressed in the presence of a point mutation (p. G608G, c.C1824T) at the locus encoding the lamin A protein. Mutations in the lamin A gene do not change the amino acid sequence necessary for protein formation, but abnormal splicing (alternative splicing) is formed in the process of mRNA transcription, resulting in an abnormal form of lamin A (progerin) with a portion of the COOH terminus of exon 11 missing (150 bp deletion). Progerin formed in this manner operates in vivo in a dominant negative manner, resulting in abnormal nuclear membrane formation at the cellular level and phenotypes associated with HGPS at the individual level (dwarfism, hair loss, skin disease, weight loss, lipodystrophy, etc.).

The term “lamin A” used herein is a type of lamin protein. Lamin protein is a protein connected to the inner membrane of the nuclear membrane and is an essential structure of the nuclear membrane, and a type of intermediate filament that plays an important role in maintaining the nuclear structure of the interphase. Like other intermediate fibers, they consist of a central alpha linear coiled-coil domain flanked by small (about 20 residues) non-linear amino terminal sequences and a larger carboxy-terminal globular domain. In addition, the lamin A protein is generated, together with the lamin C protein, from a single primary transcription of the lamin A gene (LMNA) by alternative splicing.

The term “progerin mRNA” used herein refers to the aforementioned progerin, that is, an mRNA encoding the abnormally expressed lamin A protein, and specifically, a point mutation on a locus encoding the lamin A protein (p. G608G, c.C1824T), specifically mRNA translated with the point mutation (p. G608G).

The term “gRNA (guide RNA)” used herein is RNA specific to target DNA, and gRNA may form a complex with a Cas protein and bring a Cas protein to target DNA. The gRNA may be a crRNA and a tracrRNA, or a gRNA in which a crRNA and a tracrRNA are linked into one.

A gRNA that hybridizes to progerin mRNA can be prepared by transcription of the polynucleotide of one of SEQ ID NOs: 1 to 5, and the gRNA may act with CRISRP/Cas13 to selectively cleave progerin mRNA. In addition, the gRNA transcribed from the nucleotide encoding one of the nucleotide sequences of SEQ ID NOs: 1 to 5 does not bind to mRNA encoding the normal lamin A protein and off-targets. Accordingly, more precise and reversible treatment can be performed.

The polynucleotide sequence of one of SEQ ID NOs 1 to 5 of the present disclosure is not only the corresponding sequence, but also a polynucleotide sequence exhibiting a homology of at least 40%, at least 60%, at least 80%, at least 90%, at least 95%, or at least 99% and may include any sequence that is capable of transcribing a gRNA that hybridizes to progerin mRNA or a gene encoding exon 11 and exon 12 of the LMNA gene, and a sequence having such homology and having a biological activity substantially equal to or corresponding to a polynucleotide of one of SEQ ID NOs: 1 to 5 is included in the scope of the present disclosure, even if some of the sequence has deletions, modifications, substitutions or additions.

Another aspect of the present disclosure is to provide a gRNA derived from a polynucleotide of one of SEQ ID NOs: 1 to 5.

As described above, the gRNA of the present disclosure can be obtained by transcribing polynucleotide of one of SEQ ID NOs: 1 to 5, hybridizes to progerin mRNA, and then acts with CRISRP/Cas13 to selectively cleave progerin mRNA.

In an embodiment of the present disclosure, the gRNA may hybridize to genes encoding exon 11 and exon 12 of LMNA gene. As described above, progerin is expressed with a point mutation (p. G608G, c.C1824T) on the genetic locus encoding the lamin A protein, and the mRNA encoding progerin is produced with a portion of the COOH terminus of exon 11 missing (150 bp deletion) due to abnormal splicing during transcription. At this time, the gRNA that hybridizes to progerin mRNA according to the present disclosure hybridizes to genes encoding exon 11 and exon 12 of the LMNA gene, and can selectively cleave progerin mRNA by acting with CRISRP/Cas13.

Another aspect of the present disclosure is to provide a composition for inhibiting the expression of progerin including: gRNA derived from a polynucleotide of one of SEQ ID NOs: 1 to 5; and Cas13 mRNA or a Cas13 protein derived therefrom.

gRNA derived from the polynucleotide of one of SEQ ID NOs: 1 to 5 is the same as described above.

The Cas13 mRNA or a Cas13 protein derived therefrom works as a CRISPR/Cas13 system together with the gRNA derived from the polynucleotide of one of SEQ ID NOs: 1 to 5. Specifically, the gRNA hybridizes to genes encoding exon 11 and exon 12 of the LMNA gene, and can selectively cleave progerin mRNA by acting with CRISRP/Cas13.

The “CRISPR/Cas13 system” is a third-generation genetic scissors consisting of Cas13 protein and single guide RNA, and is an artificial restriction enzyme designed to cleave a desired gene sequence using the clustered regularly interspaced short palindromic repeats (CRISPR) system, also known as the microbial immune system. Specifically, the CRISPR/Cas13 system is part of the adaptive immune system of bacteria that specifically removes foreign genes, mainly RNA, following infection by bacterial phages. It is known that effector protein Cas13 and complementary nucleotide sequences targeting external viruses, known as crRNA (crisprRNA), are expressed from the region known as the CRISPR cluster in bacterial genomes. It is known that Cas13 and crRNA combine to bind to viral RNA and have the function of cleaving the target, thereby eliminating the same. They are known as an adaptive immune system that recognizes and responds to various target RNA genes. By designing crRNA molecules that include a nucleotide sequence complementary to the target RNA, the expression of the target RNA can be controlled when delivered into higher cells with the CRISPR-Cas13 protein.

The term “Cas13 protein” used herein is an essential protein element in the CRISPR/Cas13 system, and consists largely of a REC domain (REC lobe) and a NUC domain (NUC lobe), respectively contributing to binding and cleavage with target mRNA. Specifically, certain amino acid residues in the HEPN domain bind to the scissile bond of the nucleic acids in the mRNA, followed by cleavage. This cleavage leads to the removal of the target RNA. The specific part of the target RNA to which the crRNA bound to CRISPR-Cas13 complementarily binds, is called a protospacer base sequence, and is a key factor recognized as a target. In accurately recognizing the target RNA, first, the crRNA stem loop structure is recognized and bound by the REC lobe of the CRISPR-Cas13 protein. The protospacer, which is a target RNA region complementary to crRNA to form a double helix, is recognized by the remaining NUC lobe and cleavage occurs. From among the CRISPR-Cas13 family, the CRISPR-Cas13a type is reported as having high target RNA cleavage efficiency and operating accurately without off-target cleavage in higher animal cells [2017, Nature, Omar O. Abudayyeh et al.]. In addition, the Cas13 protein can solve the problems of the conventional Cas9 protein (inability to distinguish between lamin A and progerin on DNA, off-target binding and irreversible irreversibility). The Cas13 protein includes Cas13a, Cas13b, and Cas13c, and may specifically be Cas13a in the present disclosure.

The present disclosure provides a recombinant expression vector including a polynucleotide sequence of one of SEQ ID NOs: 1 to 5 encoding a gRNA that hybridizes to progerin mRNA or a sequence complementary thereto.

The sequence of polynucleotide of one of SEQ ID NOs: 1 to 5 encoding the gRNA that hybridizes to progerin mRNA is the same as described above.

The “recombinant expression vector” refers to a recombinant DNA molecule containing a desired coding sequence and an appropriate nucleic acid sequence essential for expressing the operably linked coding sequence in a specific host organism. Promoters, enhancers, termination signals and polyadenylation signals available in eukaryotic cells are known.

In an embodiment of the present disclosure, the recombinant expression vector may further include a polynucleotide sequence encoding Cas13.

The polynucleotide sequence encoding Cas13 refers to a sequence encoding the mRNA or protein of Cas13.

According to the present disclosure, progerin mRNA is removed using genetic scissors that specifically act on progerin mRNA using the CRISPR/Cas13 system. Specifically, compared to the gene encoding the normal lamin A protein, the gene encoding the abnormal lamin A protein (progerin) undergoes a point mutation. As a result, while being deleted by 150 bp, the end of exon 11 in the expressed mRNA sequence of the progerin gene, is expressed together with exon 12. At this time, the boundary nucleotide sequence of exon 11 end and exon 12 of progerin mRNA is different from that of normal mRNA. Therefore, when a complementary sgRNA corresponding to the mutated sequence is prepared and delivered into the cells of an HGPS patient together with CRISPR/CAS13, only progerin mRNA by the mutated gene can be selectively removed.

The expression vector of the present disclosure may further include a promoter operably linked to the polynucleotide sequence.

The term “operably linked” used herein refers to a functional linkage between a gene expression regulatory sequence and another nucleotide sequence. The gene expression regulatory sequence may be one or more selected from the group consisting of a replication origin, a promoter, and a terminator. The terminator may be a polyadenylation sequence (pA), and the replication origin may be an fl replication origin, an SV40 replication origin, a pMB1 replication origin, an adeno replication origin, an AAV replication origin, or a BBV replication origin, but is not limited thereto.

The term “promoter” used herein refers to a region of DNA upstream from a structural gene, and refers to a DNA molecule to which RNA polymerase binds to initiate transcription.

A promoter according to an embodiment of the present disclosure is one of the transcription regulatory sequences that regulate the initiation of transcription of a specific gene, and may be a polynucleotide fragment with a length of about 100 bp to about 2500 bp. A promoter may be any that can regulate transcription initiation in a cell, e.g., a eukaryotic cell (e.g., a plant cell, or an animal cell (e.g., a mammalian cell such as a human, mouse, etc.). For example, the promoter may be a cytomegalovirus (CMV) promoter (e.g., human or mouse CMV immediate-early promoter), U6 promoter, elongation factor 1-a (EF1-alpha) promoter, EF1-alpha short (EFS) promoter, SV40 promoter, adenovirus promoter (major late promoter), pL λ promoter, trp promoter, lac promoter, tac promoter, T7 promoter, vaccinia virus 7.5K promoter, HSV tk promoter, SV40E1 promoter, respiratory syncytial virus (RSV) promoter, metallothionein promoter, β-actin promoter, ubiquitin C promoter, human interleukin-2 (IL-2) gene promoter, human lymphotoxin gene promoter, and human GM-CSF (human granulocyte-macrophage colony stimulating factor) may be selected from the group consisting of gene promoters, and is not limited thereto.

The recombinant expression vector according to an embodiment of the present disclosure may be selected from the group consisting of viral vectors such as plasmid vectors, cosmid vectors and bacteriophage vectors, adenoviral vectors, retroviral vectors, and adeno-associated viral vectors. Vectors that can be used as recombinant expression vectors, may be constructed based on plasmids used in the art (e.g., pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, etc.), phages (e.g., λgt4λB, λ-Charon, λΔz1, M13, etc.), or viral vectors (e.g., adeno-associated virus (AAV) vectors, etc.), and are not limited thereto.

The recombinant expression vector of the present disclosure may further include one or more selectable markers. The marker is a nucleic acid sequence having characteristics that can be selected by conventional chemical methods, and includes all genes capable of distinguishing transfected cells from non-transfected cells. For example, herbicide resistance genes such as glyphosate, glufosinate ammonium or phosphinothricin; or antibiotic resistance genes such as ampicillin, kanamycin, G418, Bleomycin, hygromycin, and chloramphenicol, may be used. However, the marker is not limited thereto.

The recombinant expression vector of the present disclosure can be prepared using gene recombination techniques well known in the art, and site-specific DNA cleavage and ligation can be performed using enzymes or the like generally known in the art.

As an example of present disclosure, the recombinant expression vector may consist of any one sequence of SEQ ID NOs: 6 to 10, wherein the sequence of any one of SEQ ID NOs: 6 to 10 of present disclosure may include not only the corresponding sequence, but also a sequence exhibiting the homology of at least 40%, at least 60%, at least 80%, at least 90%, at least 95%, or at least 99%, in which any sequence that has a biological activity substantially equal to or corresponding to a polynucleotide of one of SEQ ID NOs: 6 to 10 is included in the scope of the present disclosure, even if some of the sequence has deletions, modifications, substitutions or additions.

As an example of present disclosure, the recombinant expression vector may consist of any one sequence of SEQ ID NOs: 11 to 15, wherein the sequence of any one of SEQ ID NOs: 11 to 15 of present disclosure may include not only the corresponding sequence, but also a sequence exhibiting the homology of at least 40%, at least 60%, at least 80%, at least 90%, at least 95%, or at least 99%, in which any sequence that has a biological activity substantially equal to or corresponding to a polynucleotide of one of SEQ ID NOs: 11 to 15 is included in the scope of the present disclosure, even if some of the sequence has deletions, modifications, substitutions or additions.

The recombinant expression vector is introduced into a target cell through a known method, and the recombinant expression vector is expressed and the sgRNA hybridized to progerin mRNA binds to progerin mRNA (specifically, exon 11 and exon 12) expressed in the target cell, wherein the Cas13 protein expressed by the recombinant expression vector acts to cleave the progerin mRNA, thereby treating HGPS.

Another aspect of the present disclosure provides a composition for inhibiting the expression of progerin including the recombinant expression vector or the recombinant expression vector and the Cas13 expression vector. The composition of the present disclosure includes, as an active ingredient: a recombinant expression vector including a polynucleotide of one of SEQ ID NOs: 1 to 5 alone; a recombinant expression vector including a polynucleotide of one of SEQ ID NOs: 1 to 5 and a polynucleotide sequence encoding Cas13; or a recombinant expression vector including a polynucleotide of one of SEQ ID NOs: 1 to 5 alone and a recombinant expression vector including a polynucleotide sequence encoding Cas13.

The composition of the present disclosure may further include any known substance that does not interfere with the objective of inhibiting progerin expression.

Another aspect of the present disclosure is to provide a pharmaceutical composition for preventing or treating Hutchinson-Gilford Progeria syndrome (HGPS) and natural aging including the recombinant expression vector as an active ingredient.

The “progeria” or “Hutchinson Gilford progeria syndrome (HGPS)” is a fatal and rare genetic disease characterized by premature aging in young children, specifically caused by point mutations in the lamin A (LMNA) genetic locus (p.G608G, c.C1824T).

The term “alleviation” includes any act of inhibiting or delaying the onset of a disease by the administration of a composition.

The “treatment” used herein includes any act by which the administration of a composition ameliorates or benefits a condition.

The pharmaceutical composition may additionally include one or more suitable additives selected from the group consisting of carriers, excipients, disintegrants, sweeteners, coating agents, expanding agents, lubricants, glossing agents, flavoring agents, antioxidants, buffers, bacteriostatic agents, diluents, dispersing agents, surfactants, and binders which are commonly used in the preparation of pharmaceutical compositions.

Specifically, carriers, excipients and diluents include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil may be used, and solid dosage forms for oral administration include tablets, pills, powders, granules, and capsules. These solid preparations may be prepared by mixing, with the composition, at least one or more excipients, for example, starch, calcium carbonate, sucrose or lactose, gelatin, etc. In addition to simple excipients, lubricants such as magnesium stearate and talc may also be used. Liquid preparations for oral administration include suspensions, oral liquids, emulsions, syrups, and the like, and various excipients, such as wetting agents, sweeteners, aromatics, and preservatives, may be included in addition to commonly used simple diluents such as water and liquid paraffin. Preparations for parenteral administration include sterilized aqueous solutions, non-aqueous solvents, suspensions, emulsions, freeze-dried preparations, suppositories, and the like. Propylene glycol, polyethylene glycol, vegetable oils such as olive oil, and injectable esters such as ethyl oleate may be used as non-aqueous solvents and suspensions. As a base material of the suppository, witepsol, macrogol, tween 61, cacao butter, laurin paper, glycerogeratin and the like may be used.

According to one embodiment of the present disclosure, the pharmaceutical composition may be administered to a subject in a conventional manner via an intravenous, intra-arterial, intraperitoneal, intramuscular, intrasternal, transdermal, intranasal, inhalation, topical, rectal, oral, intraocular, or intradermal route.

The dosage of the pharmaceutical composition may vary depending on the condition and body weight of the subject, the type and severity of the disease, the drug form, and the route and duration of administration, and may be appropriately selected by a person skilled in the art. According to an embodiment of the present disclosure, but not limited thereto, the daily dosage may be 0.01 mg/kg to 200 mg/kg, 0.1 mg/kg to 200 mg/kg, or 0.1 mg/kg to 100 mg/kg. The dosage may be administered once a day or may be divided into multiple dosages, and the scope of the present disclosure is not limited thereby.

The present disclosure provides a method of alleviating or treating HGPS and natural aging including administering the composition to a subject in need thereof.

Another aspect of the present disclosure is to provide use of the composition for alleviating or treating HGPS and natural aging.

Another aspect of the present disclosure is to provide use of the composition for producing a pharmaceutical composition for alleviating or treating HGPS and natural aging.

### MODE OF DISCLOSURE

Hereinafter, one or more specific examples will be described in more detail through examples. However, these examples are intended to illustrate one or more specific examples, and the scope of the present disclosure is not limited to these examples.

### Experimental Example 1: Selection of sgRNA Hybridizing to Progerin mRNA

An sgRNA (specifically, crRNA) that hybridizes to mRNA encoding progerin, which causes HGPS, was selected.

Specifically, the sgRNA nucleotide sequence targeting progerin targets the region that is abnormally fused with exon 12 due to a mutation in exon 11, which is the nucleotide sequence that is distinct from normal lamin A. This nucleotide sequence is designed for Cas13a, for which 5 target sgRNA were selected based on their high cleavage efficiency and absence of off-target effects using two websites, CHOPCHOP (chopchop.cbu.uib.no/) and CRISPR-RT (bioinfolab.miamioh.edu/CRISPR-RT/interface/C2c2.php).

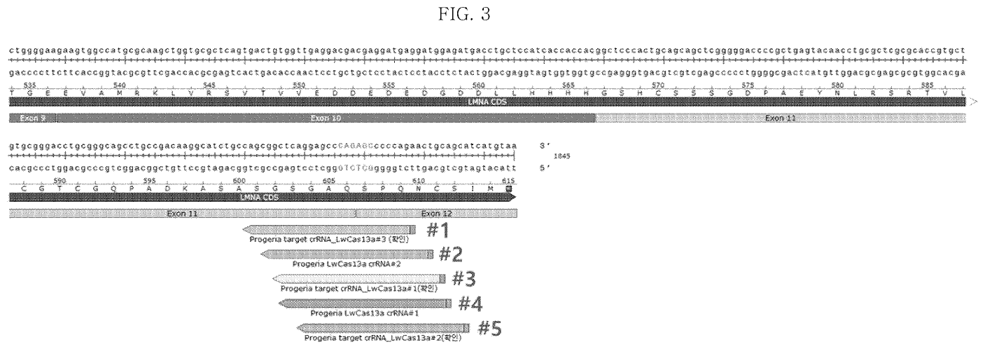

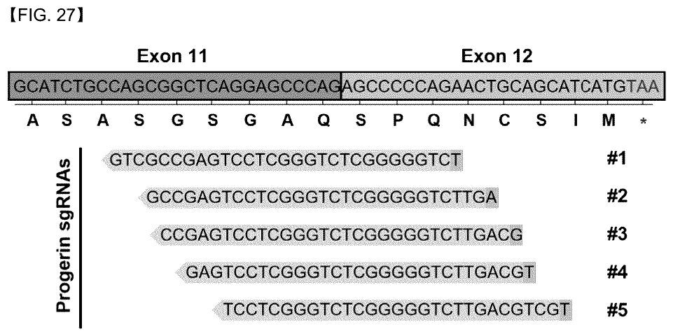

As a result, as shown in FIGS. 3 and 27, sgRNAs hybridizing to exons 11 and 12 in progerin mRNA were selected, and it was confirmed that the nucleotide sequences of SEQ ID NOs: 1 to 5 were the nucleotide sequences encoding the sgRNAs.

### Example 1: Preparation of Recombinant Expression Vector

A recombinant expression vector containing sgRNA hybridizing to exons 11 and 12 of progerin mRNA identified in Experimental Example 1, a nucleotide sequence encoding Cas13, and a promoter, was prepared.

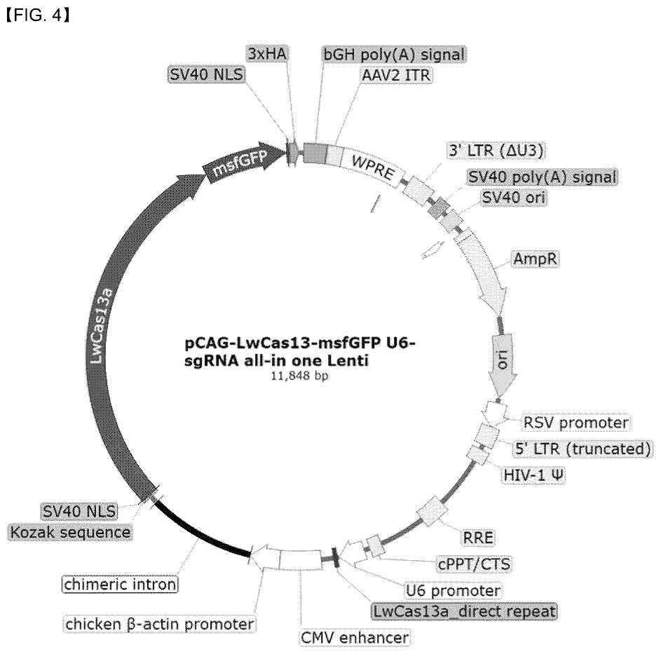

Specifically, as a backbone vector,
an all-in one backbone lentiviral vector in which Cas13a and sgRNA are simultaneously expressed, was created by cloning each required region and reconstructing them within the vector. The backbone vector was constructed by an oligo synthesis using the CMV enhancer/chicken β-actin promoter/chimeric intron (CAG) promoter and the Cas13a gene and the U6 promoter and the BsmBI restriction enzyme sequence and the spacer sequence required to insert the target sgRNA into a vector for lentiviruses (using LTR, HIV-1 Ψ, cPPT/CTS, and RRE components)(see FIG. 4).

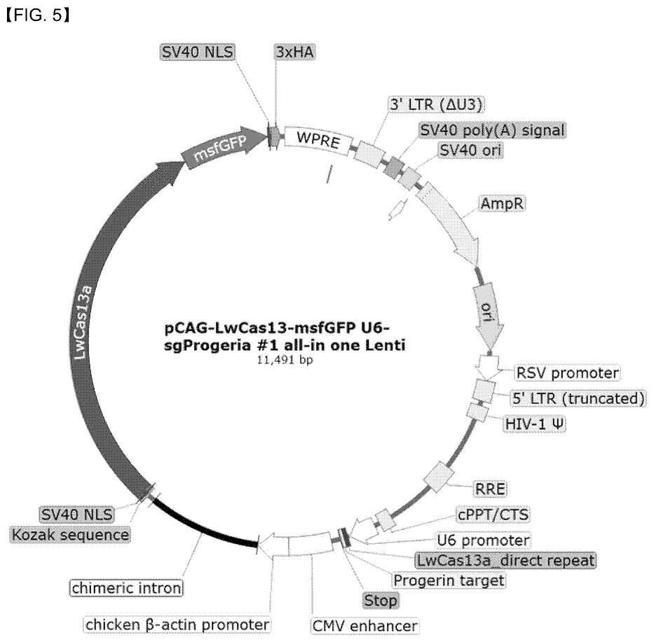

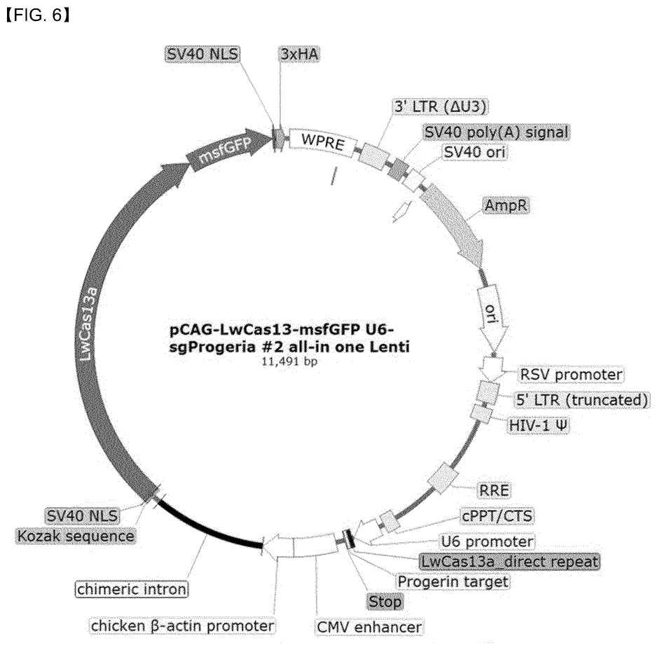

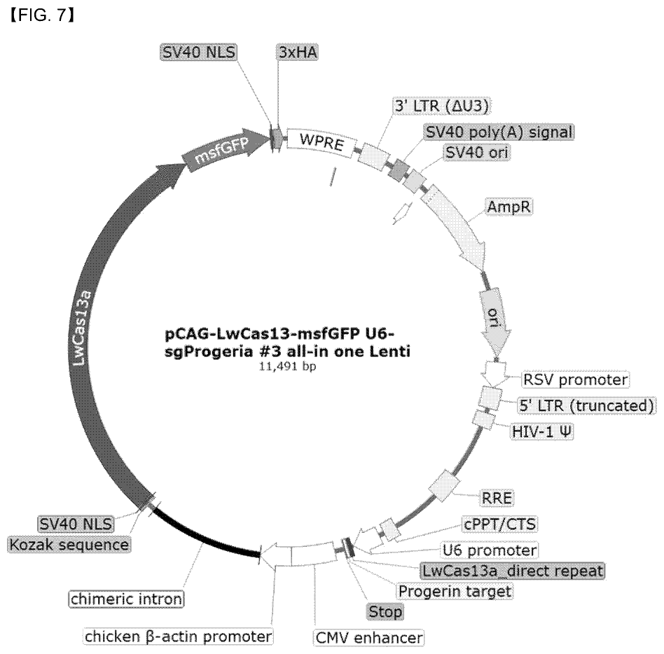

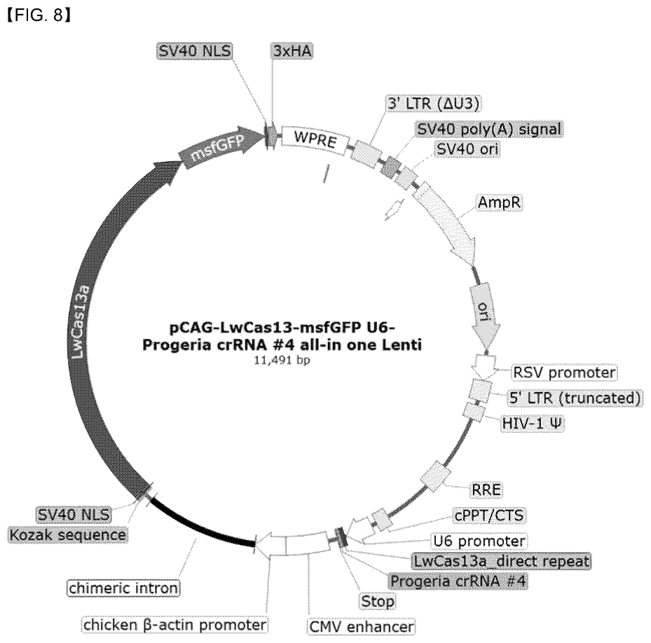

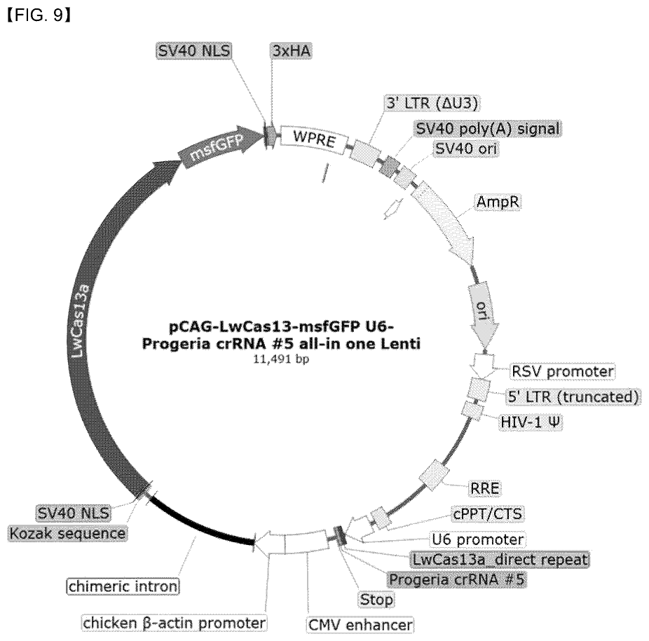

A vector was constructed by cloning the selected 5 sgRNA nucleotide sequences prepared by oligo synthesis into the BsmBI site of the backbone vector, and it was verified that the target sgRNAs were correctly entered using Sanger's sequencing method (see FIGS. 5 to 9).

As a result, the recombinant expression vectors shown in FIGS. 5 to 9 were prepared, which consist of sequences of SEQ ID NOs: 6 to 10.

### Experimental Example 2: Confirmation of Progerin mRNA and Progerin Protein Reduction Effect of Recombinant Expression Vector in Present Disclosure (In Vitro)

### Experimental Example 2-1. Construction of Normal Lamin a and Progerin Hyperexpression Vectors

In order to confirm the effect of HGPS treatment at the cellular level, a normal lamin A vector and a progerin overexpression vector were prepared. Specifically, cells do not grow well and die when progerin is overexpressed therein. Accordingly, to create a stable cell line, a conditional system (Conditional Cre-loxP) was used to make sure that progerin is expressed only when treated with 4-Hydroxytamoxifen (4-OHT).

Specifically, the LMNA overexpression vector and the progerin overexpression vector were cloned by oligo synthesis of the Loxp-RFP-2A-puro-3× poly-Loxp base sequence so that they could be expressed conditionally, and IRES was used to confirm conditional expression of the target gene (internal ribosome entry site), and into the self-made backbone vector, human lamin A (LMNA) and progerin in the coding sequence (CDS) region where genes are expressed, were cloned to construct a conditional overexpression vector.

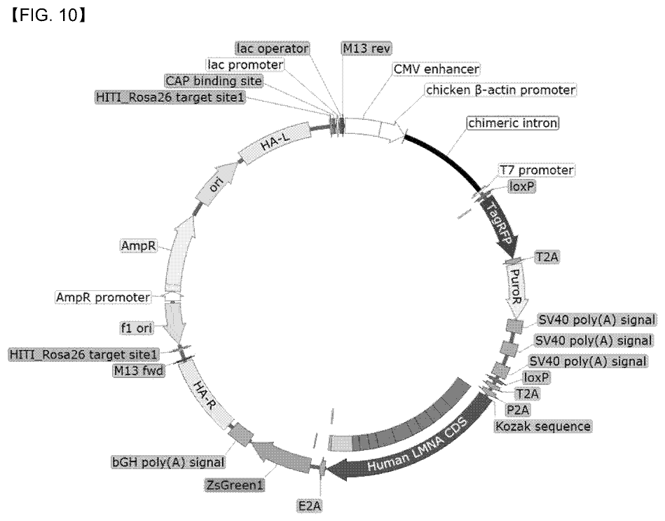

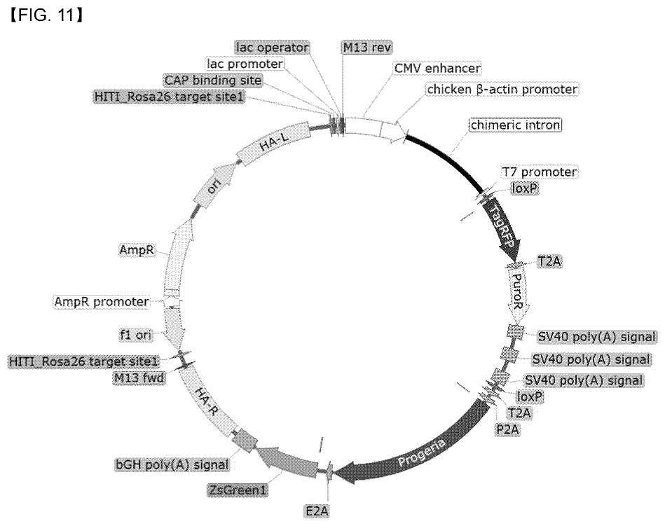

As a result, the normal lamin A (LMNA) vector and the progerin overexpression vector respectively having the structures of FIGS. 10 and 11 were constructed.

### Experimental Example 2-2. Construction of Normal Lamin a and Progerin Overexpressing Cell Lines

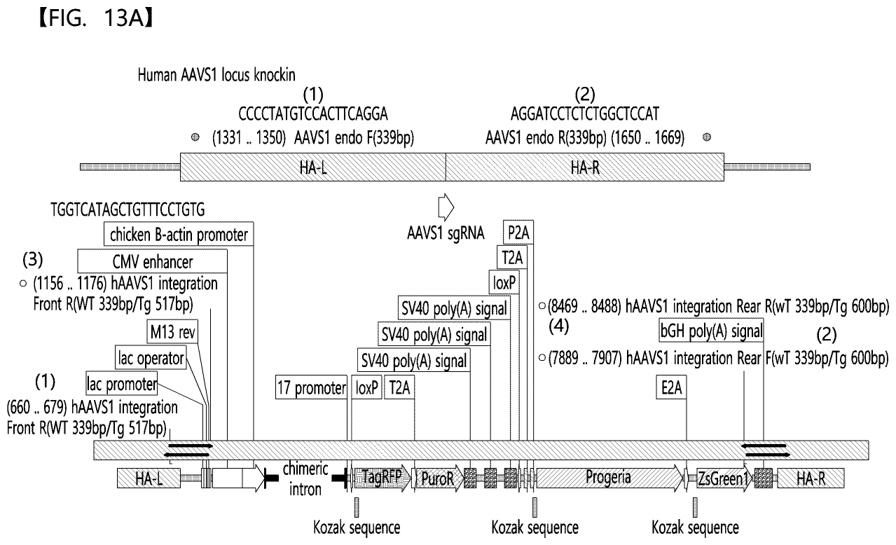

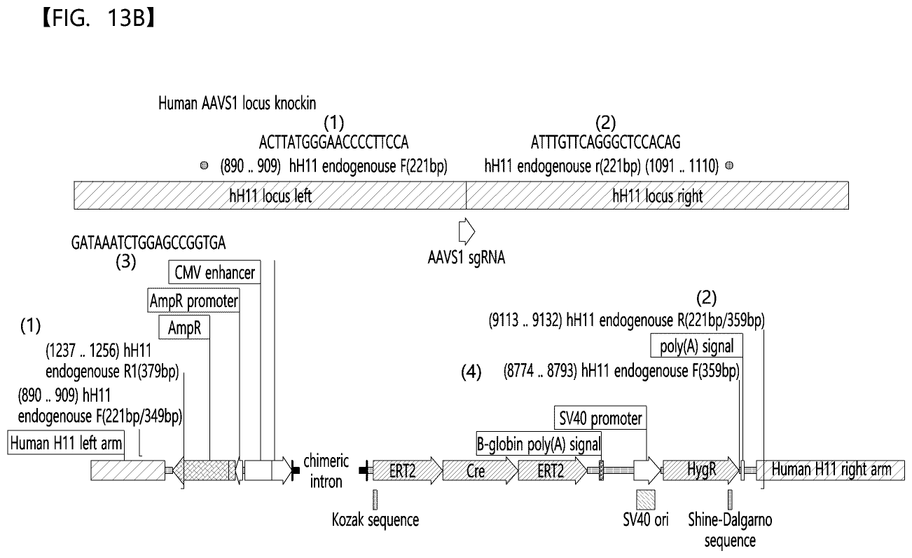

A normal lamin A and progerin overexpression cell lines were constructed using the normal lamin A and progerin overexpression vectors prepared in Experimental Example 2-1, by which the effect of the recombinant vector of the present disclosure was able to be confirmed (FIGS. 13A and 13B).

Specifically, to construct cell lines conditionally overexpressing Lamin A and progerin, each of the above-described overexpression vectors was inserted into HEK293 cells at the AAVS1 locus using genetic scissors (sgRNA sequence: GTGAGAGTTATCTGACCGTAAGG, SEQ ID NO: 16), and the ERT2CreERT2 conditional vector, which conditionally regulates Lamin A and progerin overexpression vectors, was inserted into the H11 locus using genetic scissors (sgRNA sequence: CAAAATAGGTTAGCCTTGCGGGG, SEQ ID NO: 17), and then, living cells, selected by hygromycin, puromycin and alive, were chosen and a single cell was grown in a 96-well. Then, each cell was verified by genotyping to verify whether the vector for overexpression and the vector for conditional control were inserted.

For specific verification, the PCR process was performed by inserting the following three primers (1), (2), and (3) for each of the AAVS1 and H11 locus.

AAVS1 WT PCR product: 339 bp
(F: CCCCTATGTCCACTTCAGGA (SEQ ID NO: 18),
R: AGGATCCTCTCTGGCTCCA (SEQ ID NO: 19))
AAVS1 Progerin/LMNA product: 517 bp
(F: CCCCTATGTCCACTTCAGGA (SEQ ID NO: 20),
R: TGGTCATAGCTGTTTCCTGTG (SEQ ID NO: 21))
H11 WT PCR product: 221 bp
(F: ACTTATGGGAACCCCTTCCA (SEQ ID NO: 22),
R: ATTTGTTCAGGGCTCCACAG (SEQ ID NO: 23))
ERT2CreERT2 product: variable product size due
to the HITI method (obtained by PCR using
ACTTATGGGAACCCCTTCCA (SEQ ID NO: 24),
R: GATAAATCTGGAGCCGGTGA (SEQ ID NO: 25)

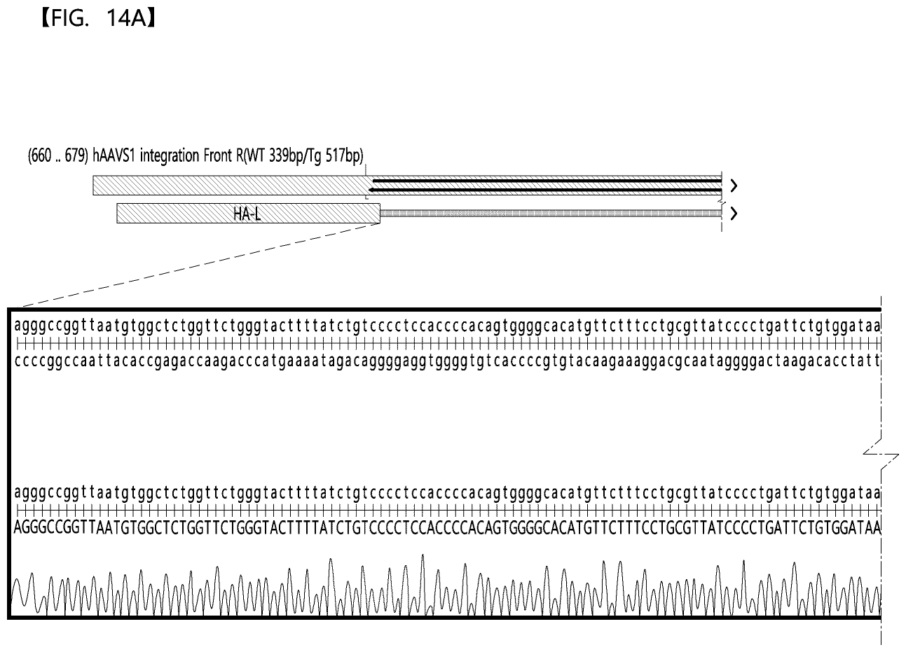

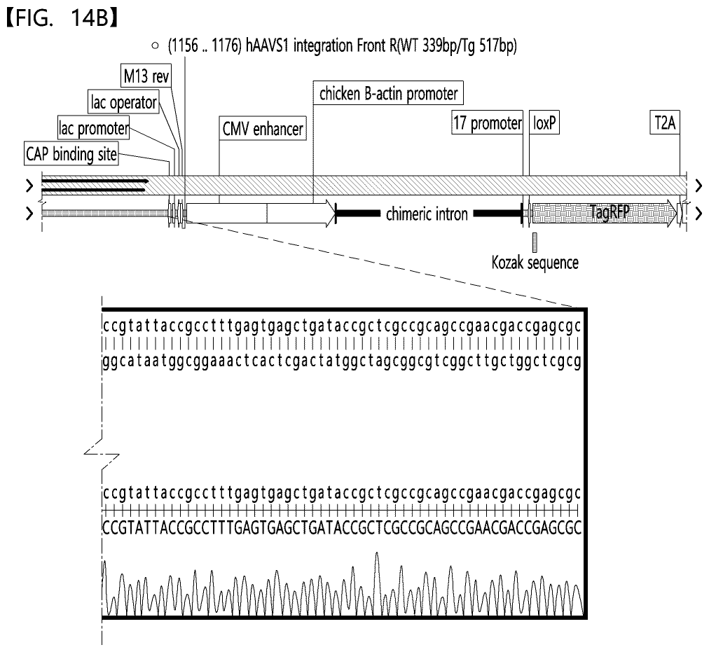

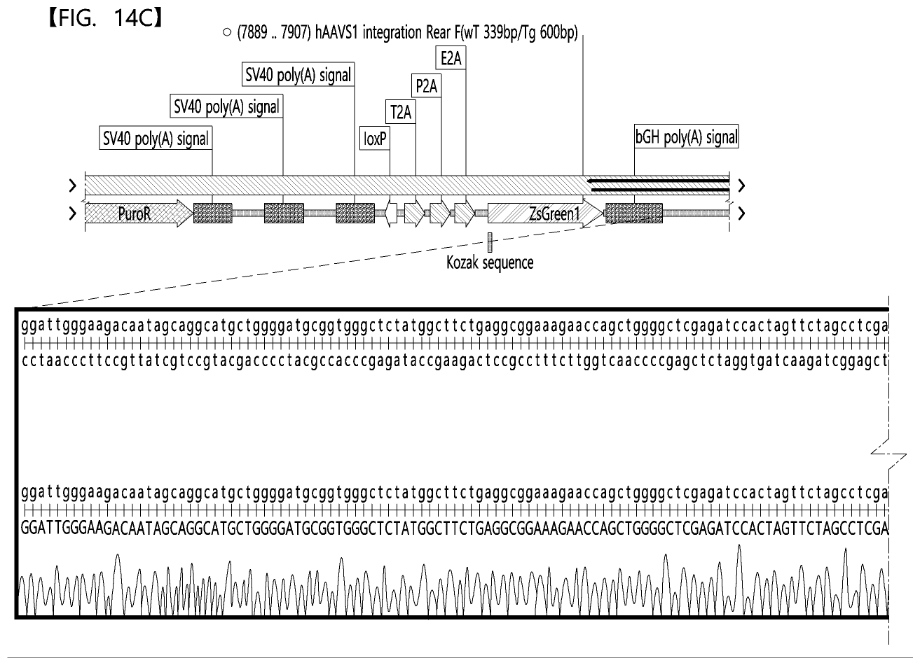

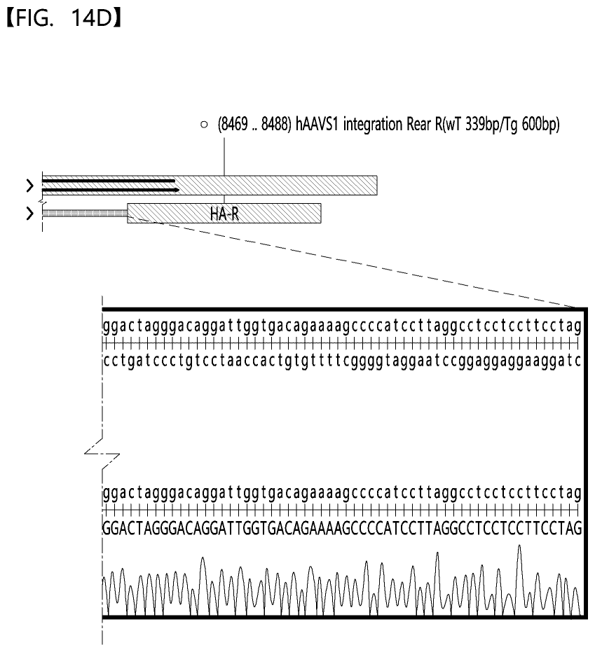

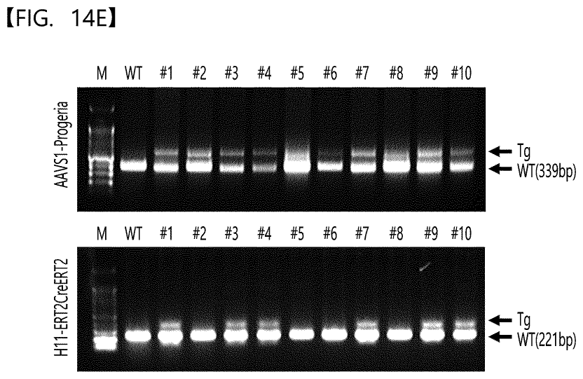

As a result, insertion of the gene was confirmed as shown in FIGS. 14A to 14E.

In addition, conditional gene expression using the inserted vector was verified through fluorescence or RT-PCR methods.

As a result, RFP was expressed before LMNA or progerin overexpressed genes were expressed, and after treatment with 4-0HT (Tamoxifen), loxp-RFP-2A-Puro-3×SV40 polyA-loxp was removed and thus, GFP was expressed together with LMNA or progerin. This verifies the conditional gene expression.

In addition, it was confirmed through semi-RT-PCR that the progerin gene was expressed after 4-0HT treatment. Primer sequences used in semi RT-PCR are shown in Table 1.

TABLE 1

| Gene | | Primer sequence | Product |
|---|---|---|---|
| Name | Accession No. | (5′→3′) | size |
| LMNA | NM_170707.4 | F: | 564 bp |
| | | CCTTGCTGACTTACC | |
| | | GGTTC | |
| | | (SEQ ID NO: 26) | |
| | | R: | |
| | | ACATGATGCTGCAGT | |
| | | TCTGG | |
| | | (SEQ ID NO: 27) | |
| Progerin | NM_001282626.2: | F: | 414 bp |
| | c.1818 + 6C > T | CCTTGCTGACTTACC | |
| | | GGTTC | |
| | | (SEQ ID NO: 28) | |
| | | R: | |
| | | ACATGATGCTGCAGT | |
| | | TCTGG | |
| | | (SEQ ID NO: 29) | |

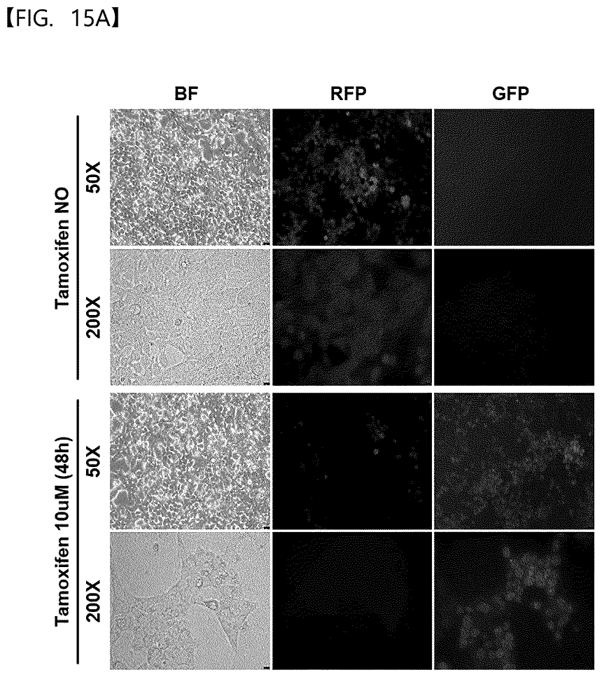

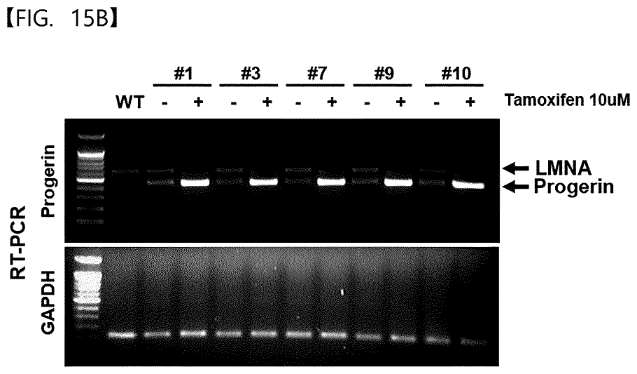

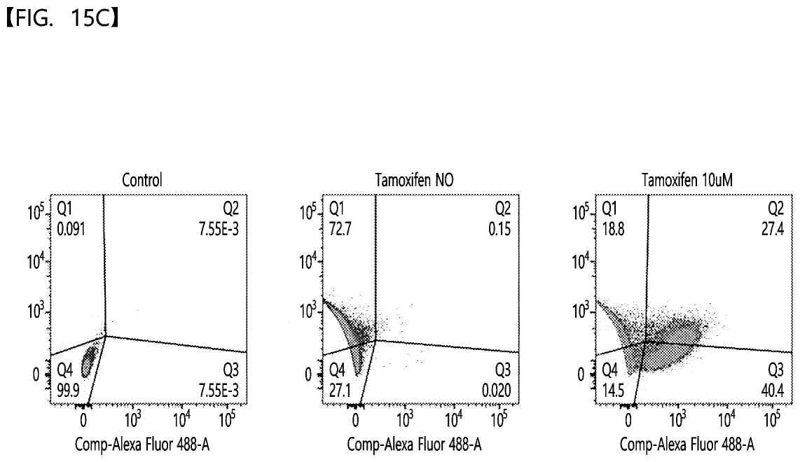

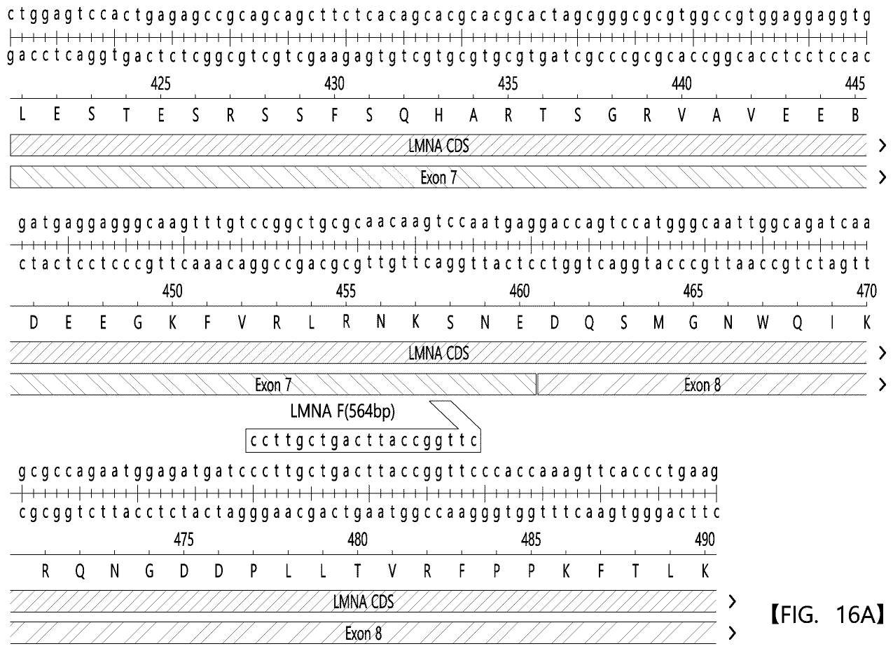

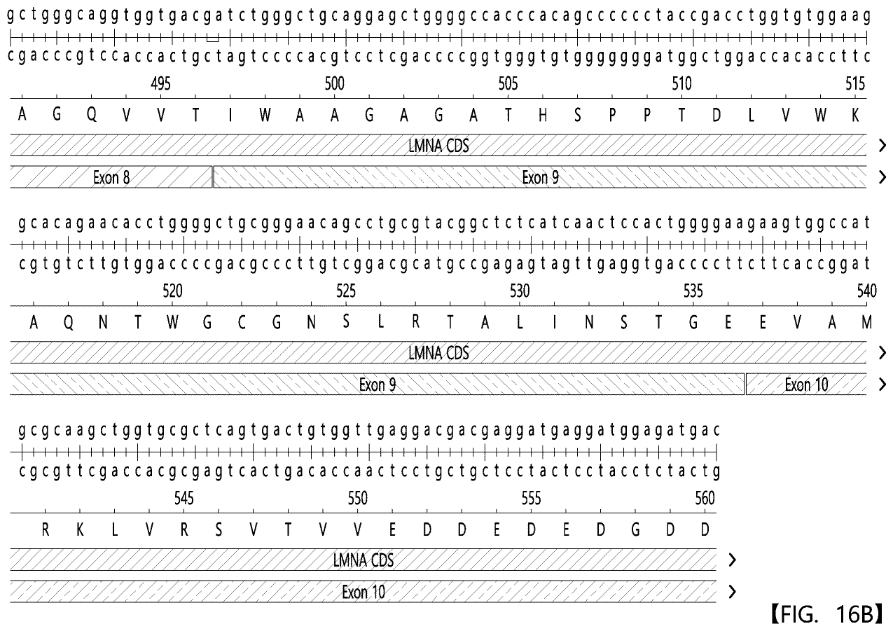

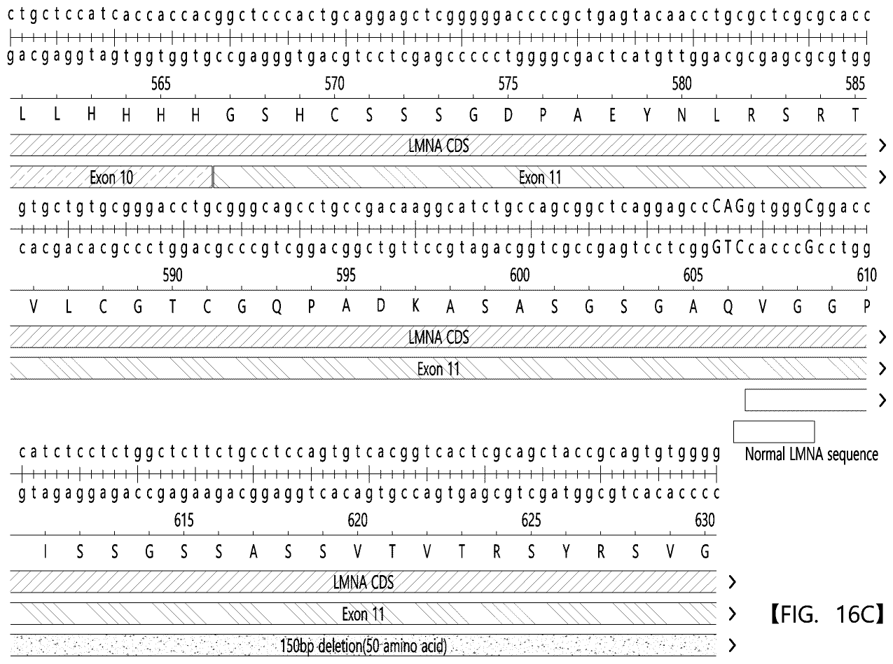

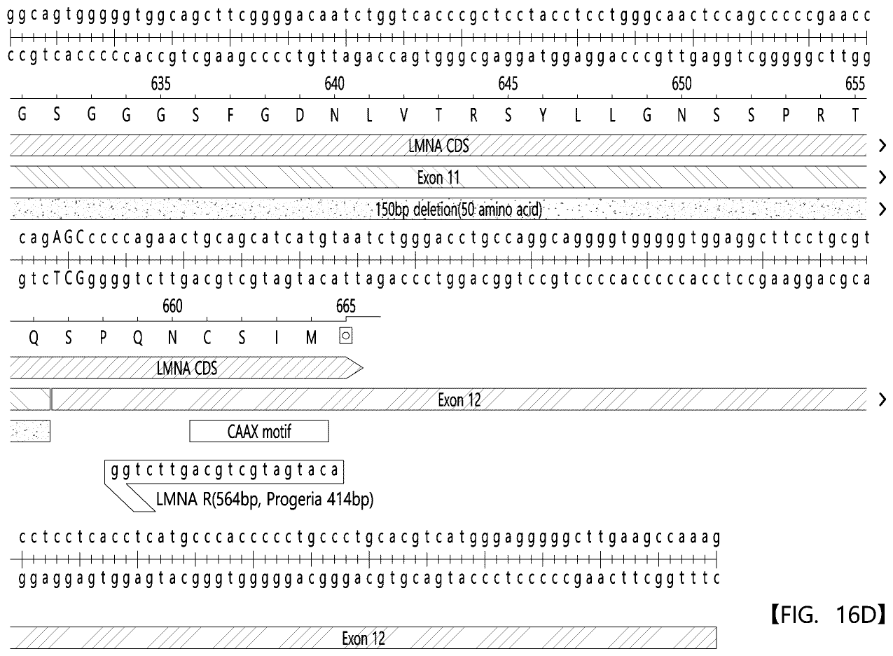

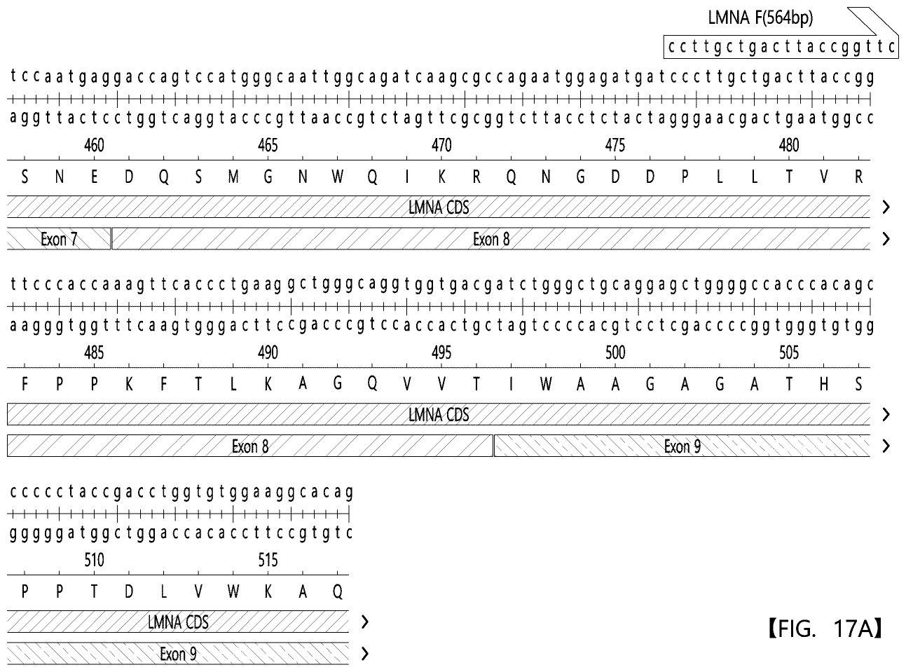

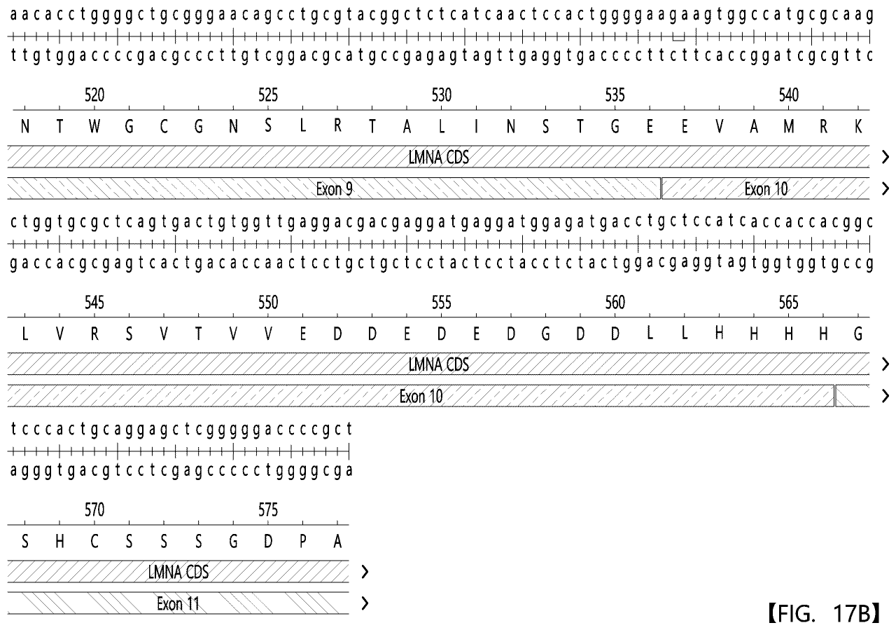

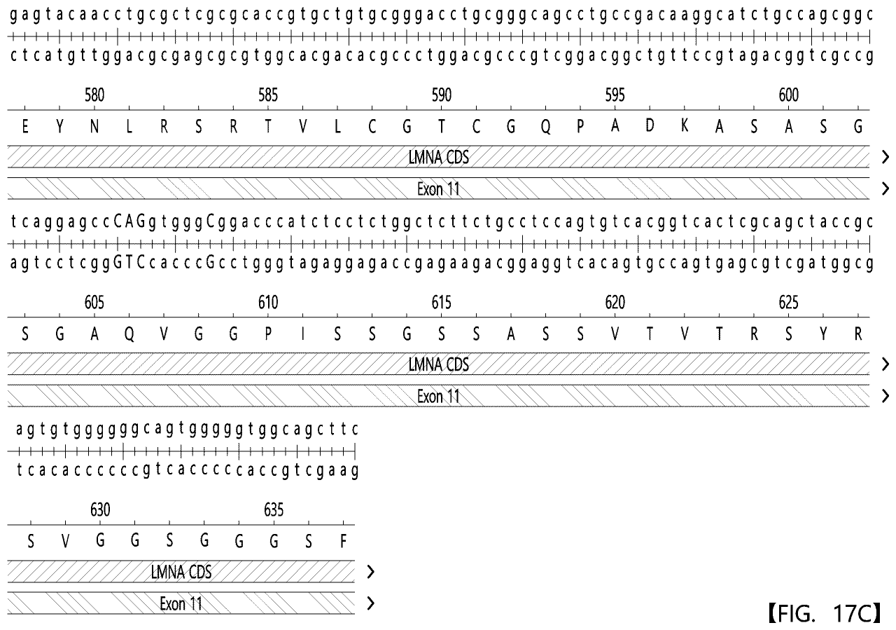

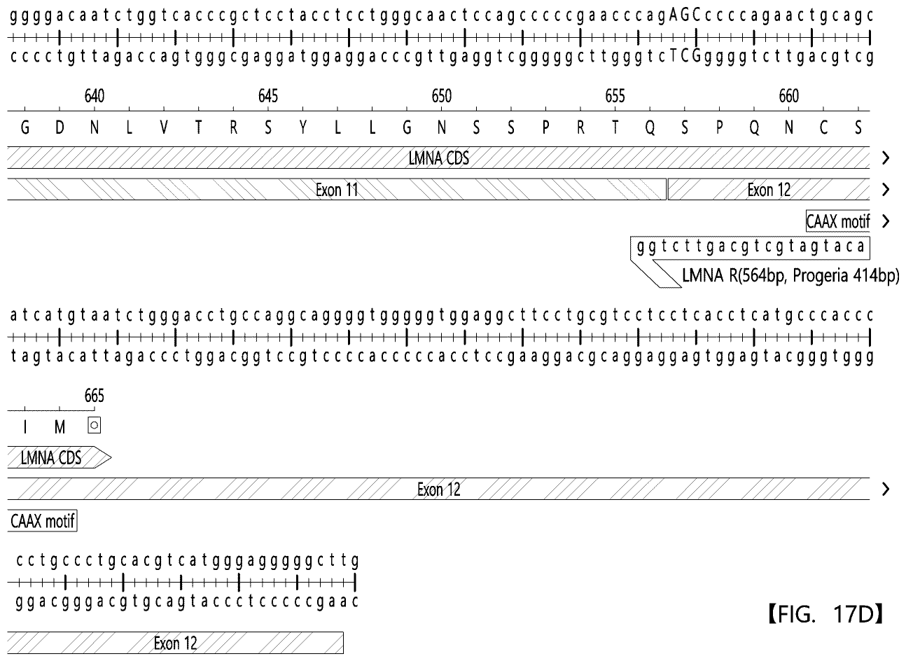

As a result, it was observed that LMNA consists of 564 base pairs (bp), while progerin, due to a mutation in exon 11, is cleaved by 150 bp, resulting in the generation of a 414 bp PCR product (see FIGS. 15A to 15C, 16A to 16D, and 17A to 17D).

Based on the above results, normal lamin A and progerin overexpressing cell lines were prepared.

### Experimental Example 2-3. Confirmation of Progerin mRNA and Protein Reduction Effect of the Recombinant Expression Vector of the Present Disclosure

The expressions of progerin mRNA and progerin protein were confirmed when the normal lamin A and progerin overexpressing cell lines, prepared in Experimental Example 2-2, were treated with the progerin overexpressing recombinant expression vector and the recombinant expression vector of the present disclosure.

Specifically, HEK293 cells, in which progerin is conditionally overexpressed, were treated with 4-OHT to induce progerin genes 24 hours before introduction of #1, #2, and #3 lentiviruses (SEQ ID NOs: 6, 7, and 8) targeting progerin. The presence or absence of overexpressed progerin was assessed 72 hours later using both RT-PCR and Western blot analysis.

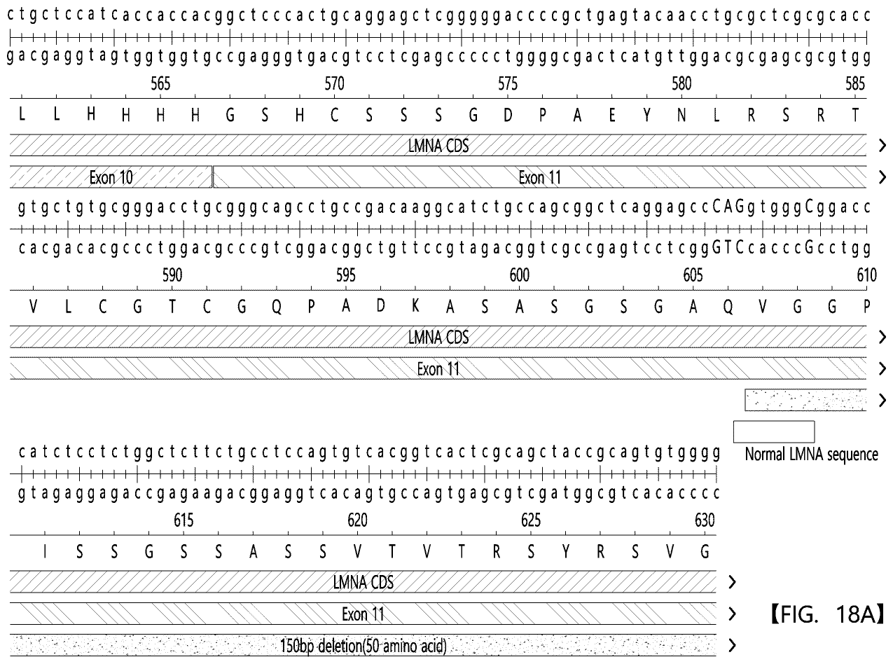

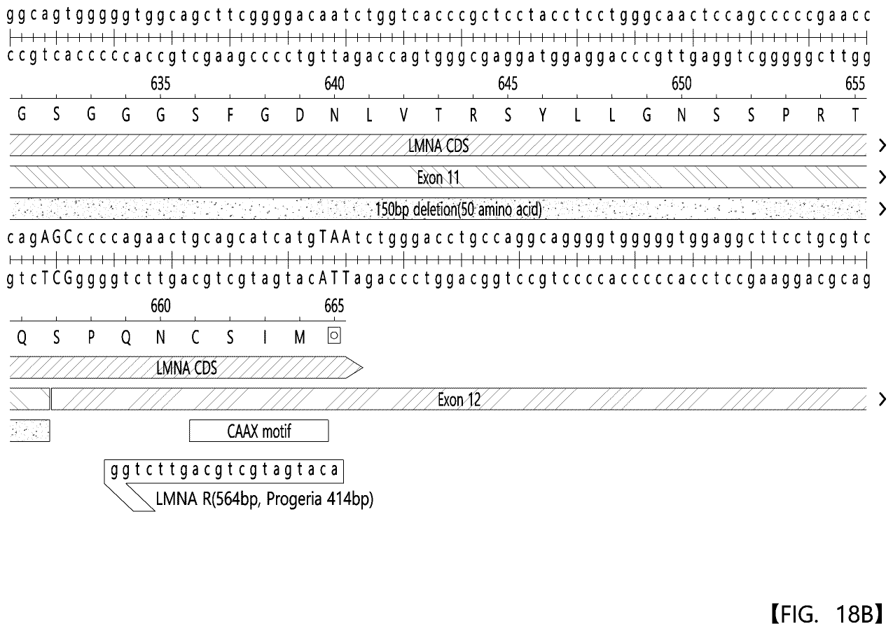

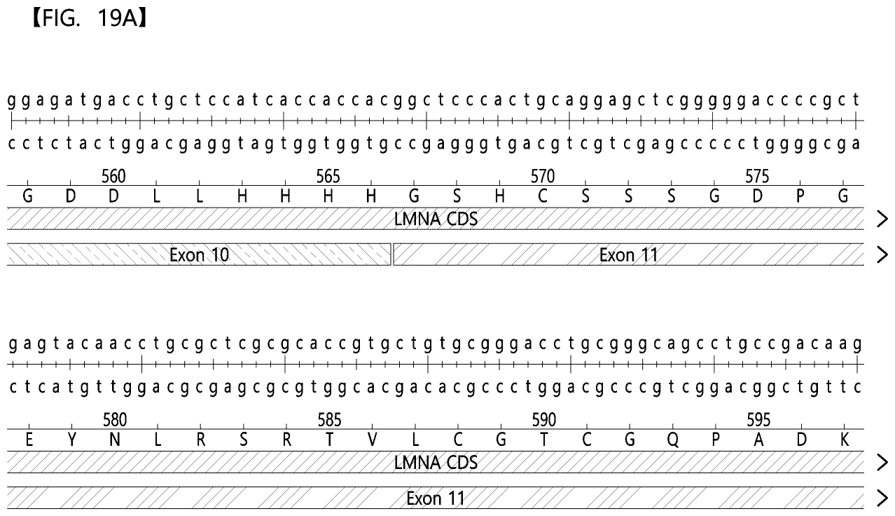

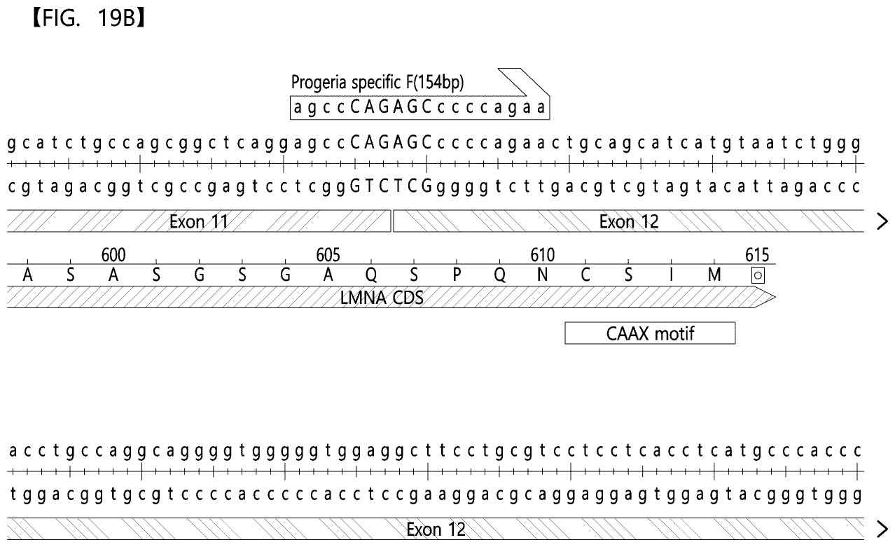

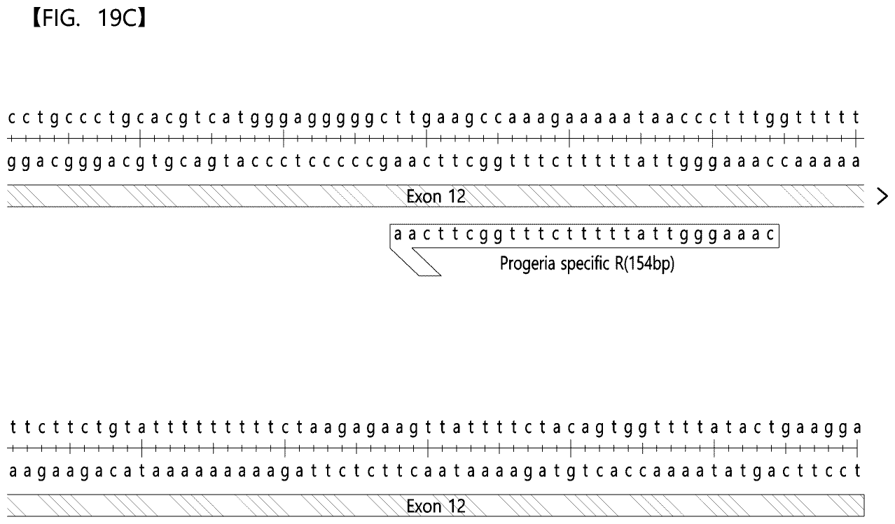

RT-PCR was performed using the primers shown in Table 2 below (FIGS. 18A, 18B and 19A to 19C).

TABLE 2

| Gene | | Primer sequence | Product |
|---|---|---|---|
| Name | Accession No. | (5′→3′) | size |
| LMNA | NM_170707.4 | F: | 148 bp |
| | | TCTGCCTCCAGTGTC | |
| | | ACG | |
| | | (SEQ ID NO: 30) | |
| | | R: | |
| | | ACATGATGCTGCAGT | |
| | | TCTGG | |
| | | (SEQ ID NO: 31) | |
| Progerin | NM_001282626.2: | F: | 154 bp |
| | c.1818 + 6C > T | AGCCCAGAGCCCCCA | |
| | | GAA | |
| | | (SEQ ID NO: 32) | |
| | | R: | |
| | | CAAAGGGTTATTTTT | |
| | | CTTTGGCTTCAA | |
| | | (SEQ ID NO: 33) | |
| GAPDH | NM_002046.7 | F: | 94 bp |
| | | TCAAGAAGGTGGTGA | |
| | | AGCAG | |
| | | (SEQ ID NO: 34) | |
| | | R: | |
| | | TCGCTGTTGAAGTCA | |
| | | GAGGA | |
| | | (SEQ ID NO: 35) | |

Regarding the progerin-specific RT-PCR primer, primer F (forward) was designed to target the region where exon 12 is joined with exon 11 that was deleted due to mutation. As a result, the PCR product is not expressed in normal LMNA and is expressed only when abnormal progerin is present.

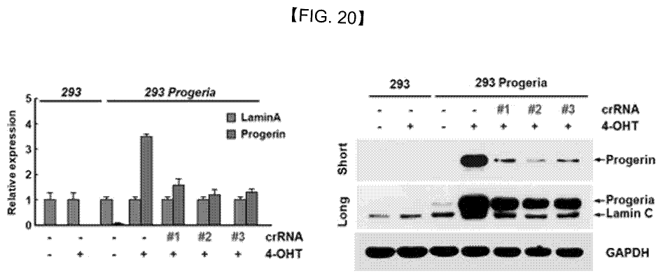

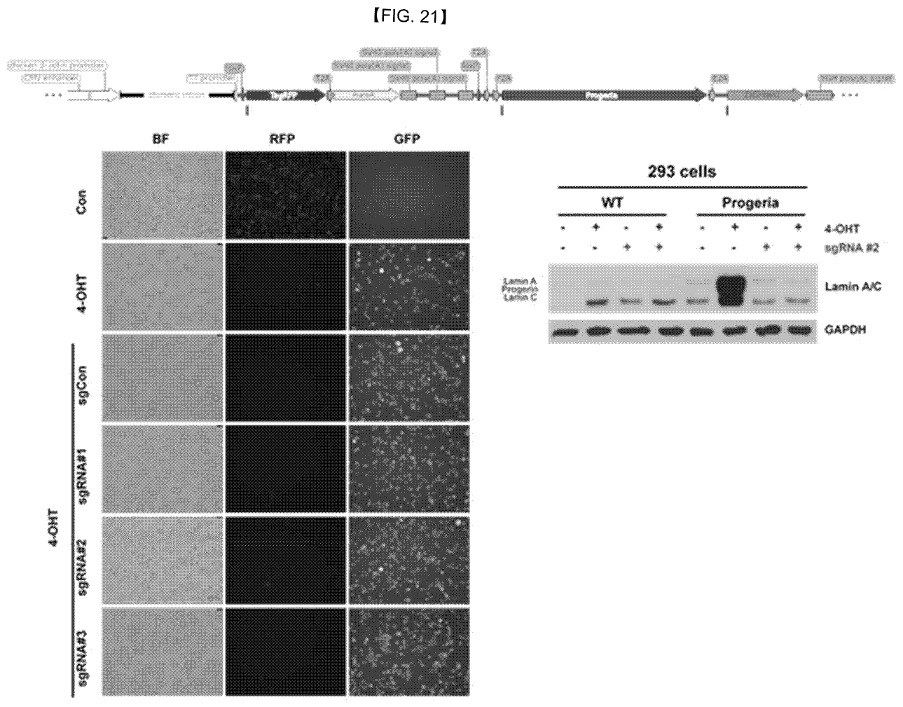

In addition, as confirmed from the above results and FIGS. 20 and 21, a certain level of expression of Lamin A was confirmed in the control HEK293 cells regardless of whether or not they were treated with 4-OHT. In addition, in the case of the experimental group introduced with the progerin overexpression vector, when not treated with 4-OHT, significant change did not occur, and even when treated with 4-OHT, there was no significant change in Lamin A expression. However, it was confirmed that the expression of progerin was increased due to the operation of the progerin vector caused by 4-OHT. On the other hand, even when the expression of progerin was induced due to the treatment using 4-OHT, in the case where the recombinant expression vectors (#1, #2 and #3, SEQ ID NOs 6, 7 and 8) of present disclosure were introduced, compared to the case where the recombinant expression vectors were not introduced, a significantly lower expression of progerin was confirmed and there was no significant difference in the expression level of lamin A. On the other hand, in the case of the HEK293 control cell line, the expression of Lamin A was not confirmed. This may be due to characteristics of the HEK293 cell line (Chromosoma. 2017, August; 126(4):501-517).

These results show that in the case of sgRNA encoded by SEQ ID NO: 2, the progerin mRNA expression level reduction effect was highest, and even when the expression of protein was checked again, similar results were obtained (FIG. 21).

### Experimental Example 3: Confirmation of Progerin mRNA and Protein Reducing Effect of the Composition of the Present Disclosure (In Vivo)

It was confirmed that the composition of the present disclosure is also effective in animals (in vivo).

### Experimental Example 3-1. Preparation of Laboratory Animals

Progerin [C57BL/6-Tg LMNA (G608G) HClns/J, stock #010667)] heterozygote (He) female and male mice purchased from Jackson lab were mated to obtain offspring. After that, wild-type (WT), heterozygote (He), and homozygote (Ho) mice were obtained through a genotyping method.

### Experimental Example 3-2. Introduction of the Composition of the Present Disclosure

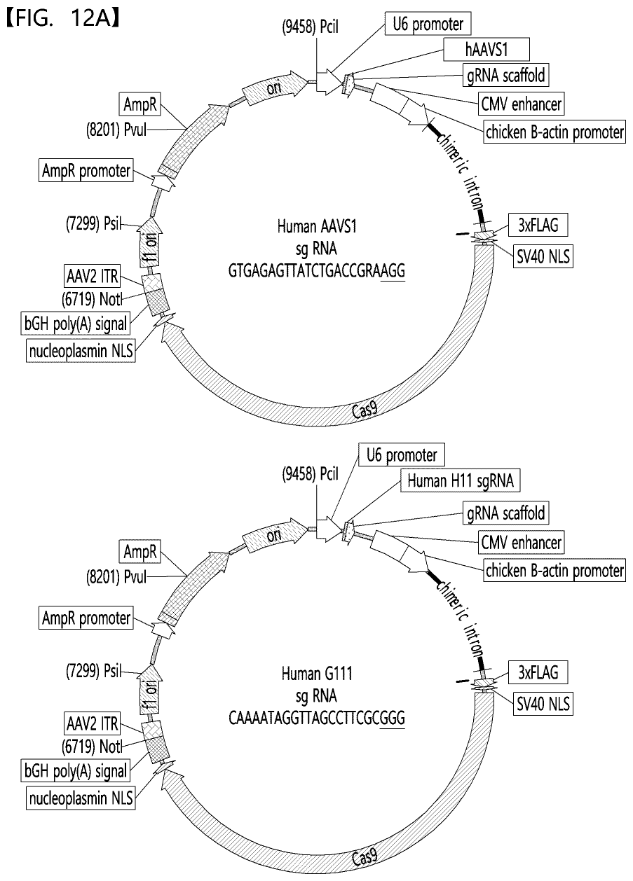

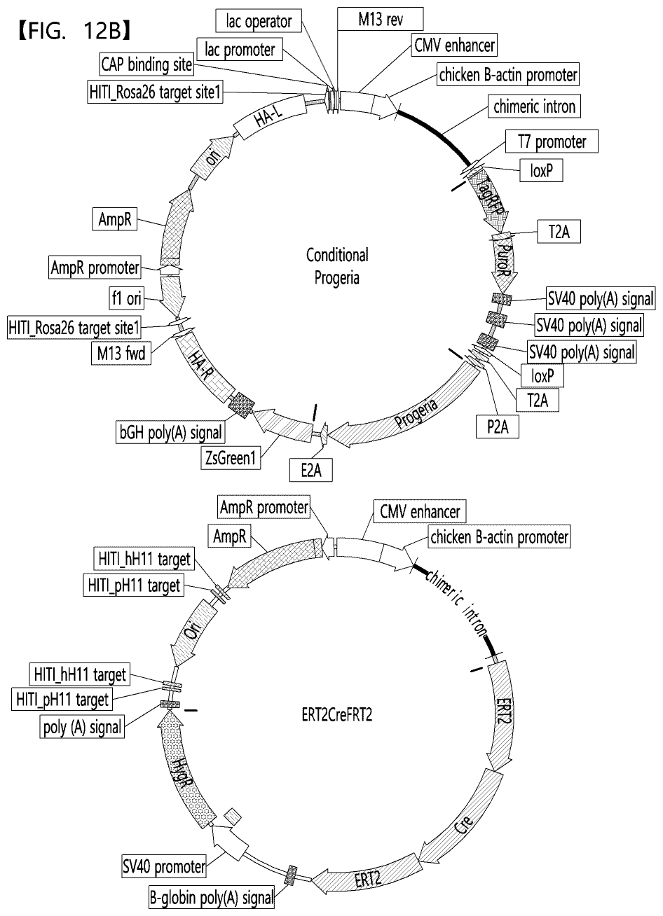

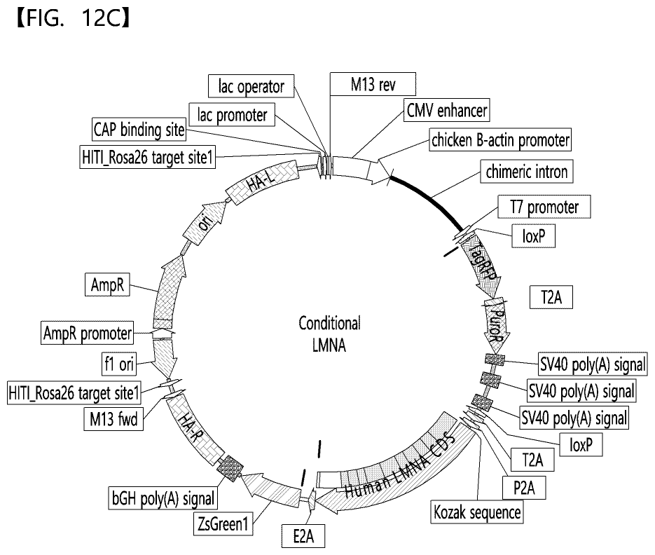

After mating heterozygous (He) female and male mice obtained in Experimental Example 3-1 with each other, mouse embryonic fibroblast (MEF) was prepared from the fetus at 13.5 days, and then only with respect to the wild type (WT) and progerin homozygote (HO) identified through genotyping methods, an experimental group (Progeria (HO) sgRNA #2 (+) in FIGS. 12A to 12C) to which the composition of the present disclosure (CRISPR-Cas13a #2 lentivirus, SEQ ID NO: 7) was introduced in the same manner as in Experimental Example 2-3, and a mouse embryonic fibroblast experimental group (Progeria (HO) sgRNA #2 (−) in FIGS. 12A to 12C) to which the composition was not introduced, were prepared.

Meanwhile, with respect to mouse embryonic fibroblasts of wild-type (WT) mice, an experimental group (WT sgRNA #2 (+) in FIG. 20) to which the composition of the present disclosure (CRISPR-Cas13a #2 lentivirus, SEQ ID NO: 7) was introduced, and a mouse embryonic fibroblast experimental group (WT sgRNA #2 (+) in FIG. 20) to which the composition was not introduced, were prepared.

### Experimental Example 3-3. Identification of Aging-Related Gene Expression

The expression of progerin and aging-related genes (PAI-1, MMP3, SMP30) of the experimental groups prepared in Experimental Example 3-2 was confirmed.

Specifically, the observation of changes in aging-related genes was confirmed through the Western blot analysis method, and the Western blot method was verified through the following processes. 30-60 μg of protein lysates were separated on 8-12% sodium dodecyl sulfate-polyacrylamide gels and transferred to 0.45 μm nitrocellulose membranes (Cat #HAWPO4700, EMD Millipore, Burlington, MA, USA). Membranes were blocked using 5% skim milk (Cat #232100, BD Biosciences) or bovine serum albumin (Cat #A9647, Sigma-Aldrich) in 10 mM Tris pH 7.4, 150 mM NaCl and 0.02% Tween-20 (TBST). Then, the resultant membranes were incubated overnight at 4° C. together with primary antibodies (Lamin A/C, Cat #4777S, Cell signaling technology, 1:1000; PAI-1, Cat #ab66705, Abcam, 1:1000; MMP3, Cat #ab52915, Abcam, 1:1000; SMP30, Cat #48864S, Cell signaling technology, 1:1000; GAPDH, Cat #LF-MA0038, AB Frontier, 1:2000). After washing the membrane three times with TBST, they were blocked with 5% skim milk in TBST and incubated for 1 hour at room temperature using a horseradish peroxidase-conjugated secondary antibody. After washing the blot with TBST, antibody binding was confirmed using SuperSignal West Pico PLUS Chemiluminescent Substrate (Cat #34580, Thermo Fisher Scientific) according to the manufacturer's instructions.

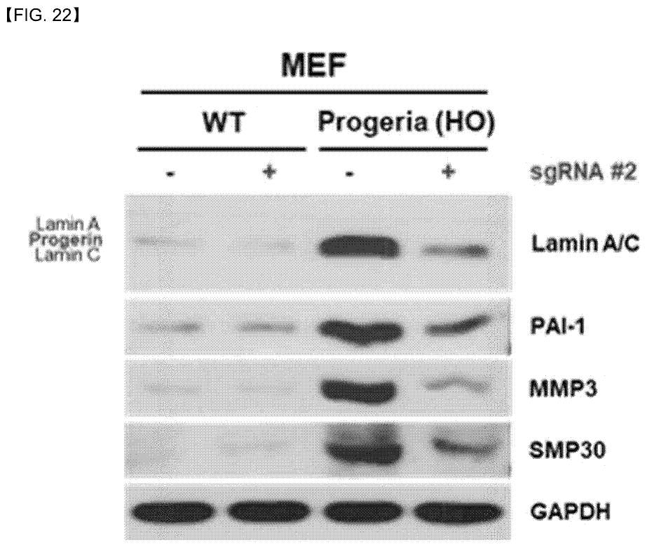

As a result, as confirmed in FIG. 22, in the case of mouse embryonic fibroblasts of wild-type mice, regardless of whether or not the composition of the present disclosure (CRISPR-Cas13a #2 lentivirus, SEQ ID NO: 7) was introduced, any significant aging-related gene expression differences (WT sgRNA #2 (−) and (+) in FIG. 20) did not occur. On the other hand, with respect to the homozygote (Ho), in the case of the mouse embryonic fibroblast experimental group (Progeria (HO) sgRNA #2 (−) in FIG. 20) to which the composition (CRISPR-Cas13a #2 lentivirus, SEQ ID NO: 7) was not introduced, the expression of progerin and aging-related genes (PAI-1, MMP3, SMP30) increased compared to the wild-type cell line. In addition, in the case of the experimental group (Progeria (HO) sgRNA #2 (+) in FIG. 20) introduced with the composition of the present disclosure (CRISPR-Cas13a #2 lentivirus, SEQ ID NO: 7) (Progeria (HO) sgRNA #2 (+) in FIG. 20), the expression of progerin and aging-related genes (PAI-1, MMP3, SMP30) was decreased compared to the experimental group to which the composition was not introduced.

### Experimental Example 3-4. Administration and Effect Verification of the Composition of the Present Disclosure

An experimental group (F4 #08 (HO, M, male) and F4 #13 (HO, F, female) in Table 3 below) prepared by adding the composition of the present disclosure (CRISPR-Cas13a #2 lentivirus, SEQ ID NO: 7) prepared in Example 1 above to the homozygous mice of Experiment 3-1; and an experimental group (F4 #01 (HO, M) and F4 #21 (HO, M) in Table 3 below) to which the composition was not added, were compared based on body weight, which is an indicator of HGPS.

Specifically, to progeria Ho mice aged 7 months (date of birth (DOB): Oct. 21, 2019, virus injection time (Apr. 24, 2020)), a control (backbone virus without target sgRNA sequence) and a virus constructed using sgRNA #2 were injected through the tail vein, and the change in body weight by progeria was measured at regular intervals from the start of the experiment (Apr. 24, 2020).

As a result, as confirmed from Table 3, the experimental group, not injected with the composition of the present disclosure, died, and the experimental group, injected with the composition of the present disclosure, showed a slight weight loss and the survival rate was increased compared to the control.

TABLE 3

| | Mice | F4 #01 (HO, M) | F4 #08 (HO, M) | F4 #21 (HO, F) | F4 #13 (MO, F) |
|---|---|---|---|---|---|
| | DOB | Oct. 21, 2019 | Oct. 21, 2019 | Oct. 21, 2019 | Oct. 21, 2019 |
| First virus | Apr. 24, 2020 | control | #2 | control | #2 |
| | (Thu) | | | | |
| Second virus | Apr. 27, 2020 | control | #2 | control | #2 |
| | (Mon) | | | | |
| Body weight | Apr. 24, 2020 | 21.8 g | 21.5 g | 17.5 g | 17.1 g |
| | Apr. 27, 2020 | 21.3 g | 21.1 g | 17.9 g | 17.5 g |
| | May 4, 2020 | 20.2 g | 214 g | 17.0 g | 16.9 g |
| | May 11, 2020 | 20.6 g | 21.2 g | 16.6 g | 16.7 g |
| | May 18, 2020 | 20.2 g | 20.5 g | 17.0 g | 17.1 q |
| | May 25, 2020 | Death (May 22, 2020) | 20.3 g | 14.9 g | 16.6 g |
| | Jun. 1, 2020 | | 19.0 g | Death (May 29, 2020) | 14.9 g |
| | | | | 12.5 g | |

Meanwhile, the expression of progerin and aging-related genes (PAI-1, MMP3, SMP30) in organ tissues (liver, lung, kidney) of the experimental groups was confirmed.

Specifically, the same method as in Experimental Example 3-3 was used except for the liver, lung and kidney samples of the mouse subject of Experimental Example 3-4.

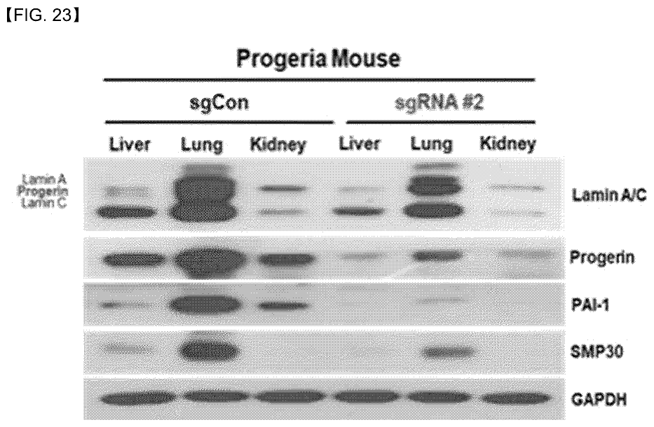

As a result, as shown in FIG. 23, progerin and aging-related gene (PAI-1, MMP3, SMP30) expression in the liver, lungs, and kidneys was reduced in the experimental group treated with the composition of the present disclosure compared to the untreated group.

### Experimental Example 4: Validation of Progerin mRNA Target Sequence

### Experimental Example 4-1. Fluorescence Analysis

To MEFs of Progeria HO mice, 293FT (Invitrogen #R70007) cells were introduced using a control and a recombinant expression vector of one of SEQ ID NOs: 6 to 10 of the present disclosure, pMD2.G (Addgene #12259), and psPAX2 (Addgene #12260) vectors. From 4 to 5 days after the introduction, MEFs of Progeria HO mice were infected twice with lentiviruses produced in the supernatant of the cell culture, and expression of CRISPR-Cas13a was confirmed by GFP fluorescence.

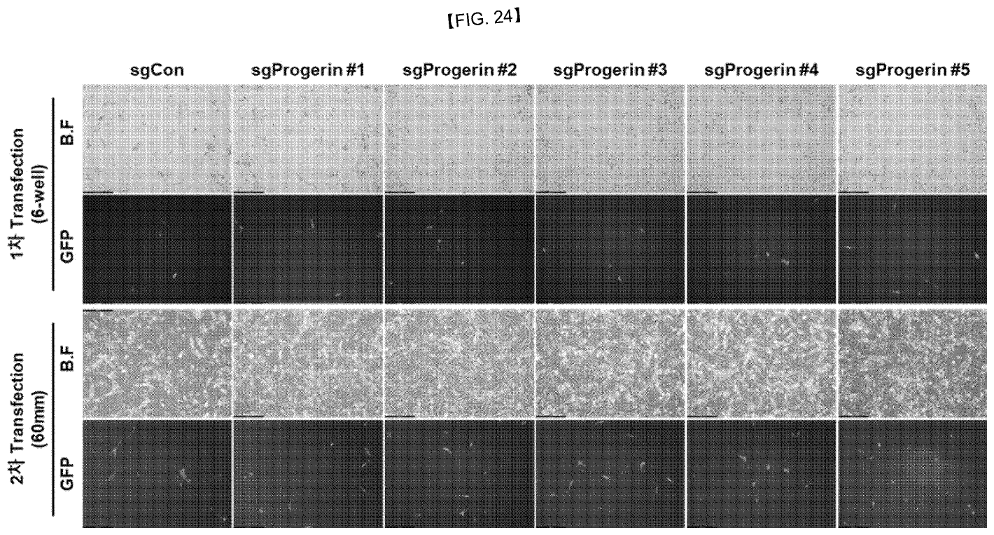

As a result, as confirmed in FIG. 24, it was confirmed that the recombinant lentivirus (CRISPR-Cas13a #1 to #5 lentivirus, SEQ ID NOs: 6 to 10) was introduced and GFP fluorescence fused with Cas13a was expressed.

### Experimental Example 4-2. Confirmation of Expression of Progerin and Aging-Related Gene

The recombinant expression vector of the present disclosure (CRISPR-Cas13a #1 or #5 lentivirus, SEQ ID NO: 6 to 10) was introduced to HEK293 cells conditionally overexpressing progerin constructed in Experimental Example 2-3 and MEFs obtained in Experimental Example 3-2 in the same manner as in Experimental Example 2-3. In addition, progerin and aging-related gene expression changes were confirmed through Western blot analysis.

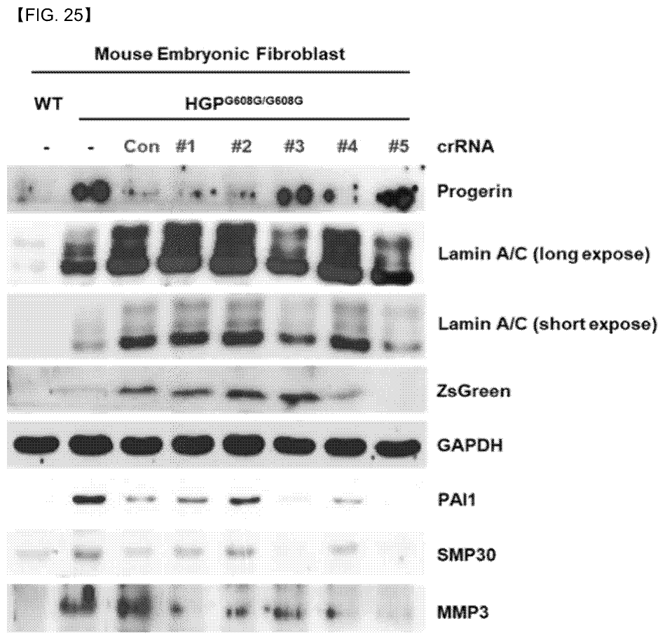

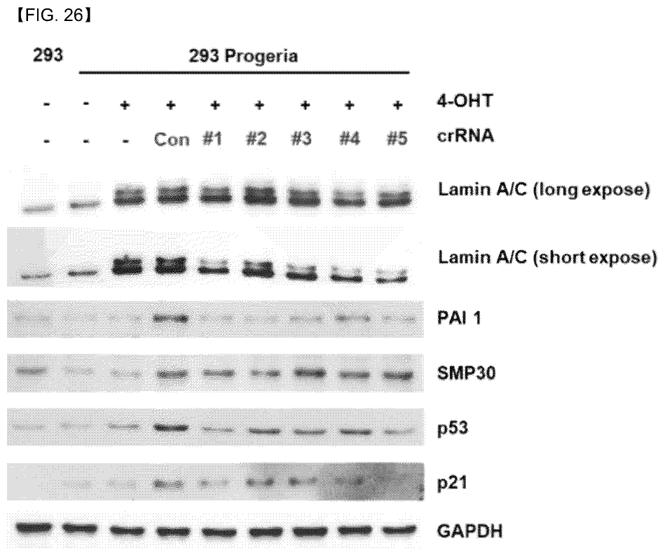

As a result, it was confirmed that the expression of progerin and aging-related genes (PAI-1, MMP3, and SMP30) decreased in the experimental group into which the recombinant expression vector of the present disclosure was introduced than in cells in which the recombinant expression vector of the present disclosure was not introduced (FIGS. 25 and 26).

### Example 2: Preparation of Recombinant Expression Vector

A recombinant expression vector containing sgRNA hybridizing to exons 11 and 12 in progerin mRNA identified in Experimental Example 1, a nucleotide sequence encoding Cas13, and a promoter was prepared.

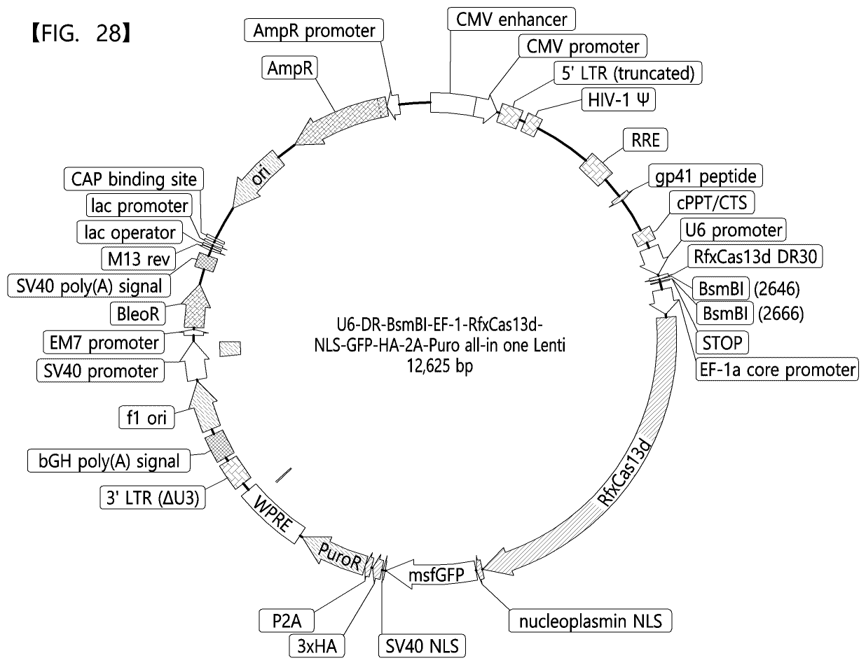

Specifically, referring to FIG. 28, as a backbone vector, an all-in one backbone lentiviral vector in which Cas13d and sgRNA are simultaneously expressed, was created by cloning each required region and reconstructing them within the vector by the research team. The backbone vector was constructed by an oligo synthesis using the CMV enhancer/chicken β-actin promoter/chimeric intron (CAG) promoter and the Cas13d gene and the U6 promoter and the BsmBI restriction enzyme sequence and the spacer sequence required to insert the target sgRNA into a vector for lentiviruses (using LTR, HIV-1 Ψ, cPPT/CTS, and RRE components).

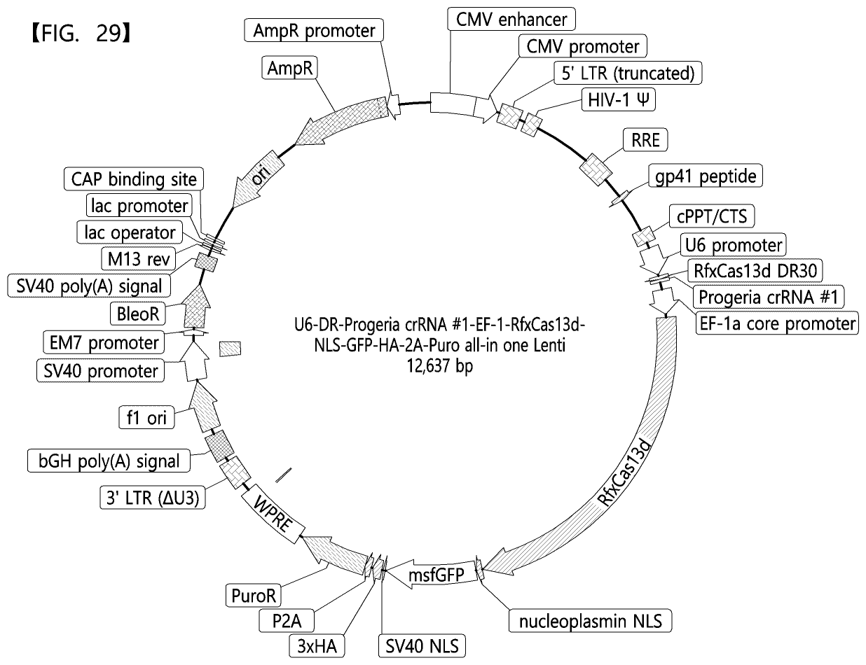

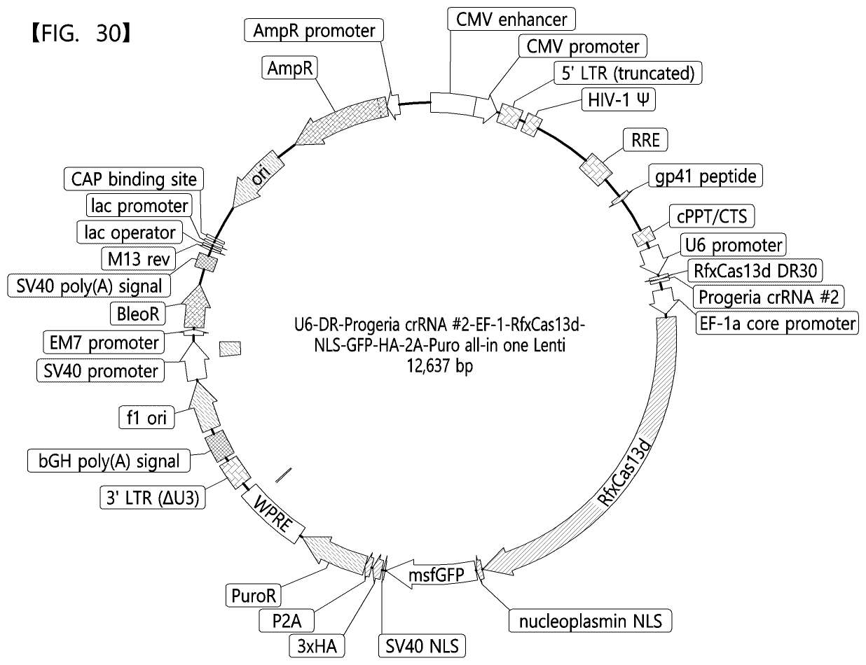

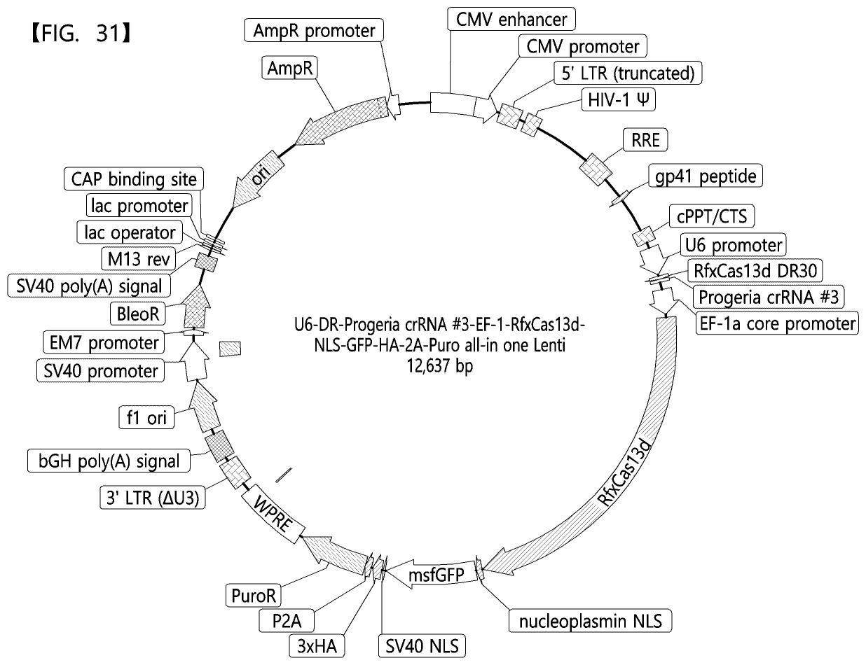

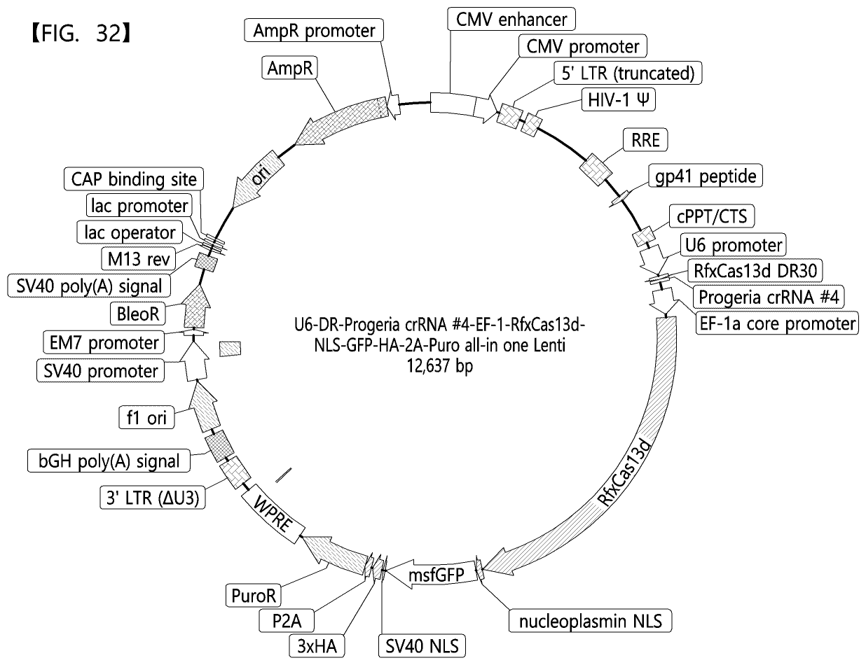

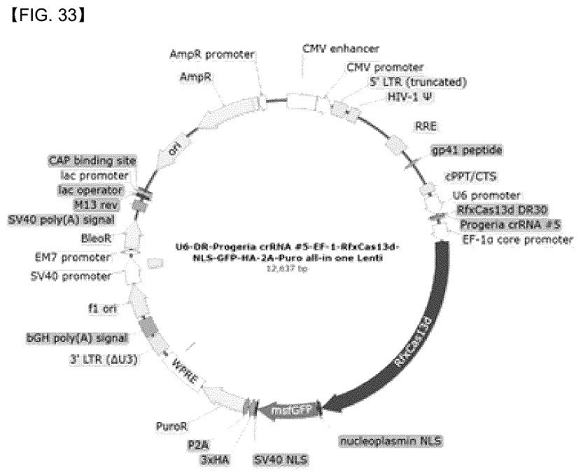

The gene therapy vectors shown in FIGS. 29 to 33 were constructed by cloning selected 5 sgRNA base sequences, prepared through oligo synthesis, into the BsmBI site of the backbone vector shown in FIG. 28, and Sanger's sequencing method was used to verify that the correct target sgRNAs were included.

As a result, the recombinant expression vectors shown in FIGS. 29 to 33 were prepared. The recombinant expression vectors consisted of the sequences of SEQ ID NOs: 11 to 15.

### Experimental Example 5: Confirmation of Progerin mRNA and Progerin Protein Reduction Effect of Recombinant Expression Vector in Present Disclosure (In Vitro)

### Experimental Example 5-1. Confirmation in Progerin Overexpressing Cell Lines

The expressions of progerin mRNA and progerin protein were confirmed when progerin overexpressing cell lines, prepared in Experimental Example 2-2, were treated with the progerin overexpressing recombinant expression vector and the recombinant expression vector of SEQ ID NOs: 11 to 15 of the present disclosure.

HEK293 cells conditionally overexpressing progerin were treated with 4-OHT 24 hours before introducing recombinant vectors (sequence numbers 11 to 15) of #1, #2, #3, #4, and #5 targeting progerin, to induce progerin genes. Then, as for whether progerin is overexpressed or not, the reduction in progerin was confirmed by RT-PCR and Western blot 72 hours after the induction of progerin genes.

RT-PCR was performed using the primers in Table 4 (Primer sequences for qRT-PCR analysis).

TABLE 4

| Gene | | Primer sequence | Product |
|---|---|---|---|
| Name | Accession No. | (5′→3′) | size |
| LMNA | NM_170707.4 | F: | 148 bp |
| | | TCTGCCTCCAGTGTC | |
| | | ACG | |
| | | (SEQ ID NO: 30) | |
| | | R: | |
| | | ACATGATGCTGCAGT | |
| | | TCTGG | |
| | | (SEQ ID NO: 31) | |
| Progerin | NM_001282626.2: | F: | 154 bp |
| | c.1818 + 6C > T | AGCCCAGAGCCCCCA | |
| | | GAA | |
| | | (SEQ ID NO: 32) | |
| | | R: | |
| | | CAAAGGGTTATTTTT | |
| | | CTTTGGCTTCAA | |
| | | (SEQ ID NO: 33) | |
| GAPDH | NM_002046.7 | F: | 94 bp |
| | | TCAAGAAGGTGGTGA | |
| | | AGCAG | |
| | | (SEQ ID NO: 34) | |
| | | R: | |
| | | TCGCTGTTGAAGTCA | |
| | | GAGGA | |
| | | (SEQ ID NO: 35) | |

Regarding the progerin-specific PCR primer, primer F (forward) was designed to target the region where exon 12 is joined with exon 11 that was deleted due to mutation. As a result, the PCR product is not expressed in normal LMNA and is expressed only when abnormal progerin is present.

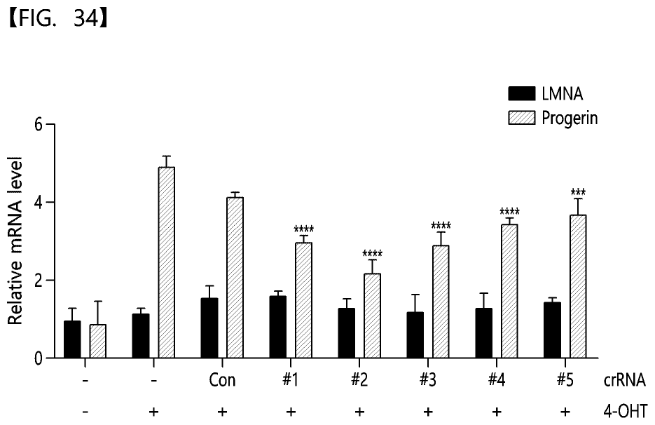

As a result, as confirmed from FIG. 34, in the case of the experimental group into which the progerin overexpression vector was introduced, there was no significant change when not treated with 4-OHT, and due to the operation of the progerin vector due to 4-OHT, the expression of progerin was increased. On the other hand, even in the case were the expression of progerin was induced by the treatment with 4-OHT, when the recombinant expression vectors (#1, #2, #3, #4 and #5) of SEQ ID NOs: 11 to 15 of the present disclosure were introduced, a significantly lower expression of progerin was confirmed compared with the experimental group into which the vectors were not introduced.

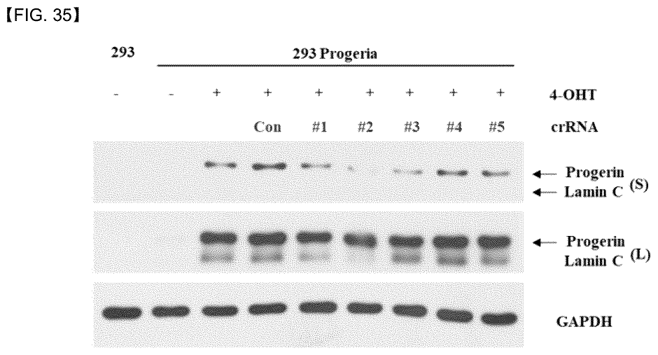

These results show that in the case of sgRNA encoded by SEQ ID NO: 2, the progerin mRNA expression level reduction effect was highest, and even when the expression of protein was checked again, similar results were obtained (FIG. 35).

### Experimental Example 5-2. Identification in Mouse Embryonic Fibroblasts

The progerin inhibitory effect of the recombinant expression vectors of SEQ ID NOs: 11 to 15 (#1, #2, #3, #4, and #5) of the present disclosure was verified in the same manner as used in Experimental Example 5-1, using the mouse embryonic fibroblasts obtained in Experimental Example 3. An experimental group into which the compositions of the present disclosure (CRISPR-Cas13d #1, #2, #3, #4, and #5, SEQ ID NOs: 11 to 15) were introduced, and an experimental group into which the compositions were not introduced, were prepared.

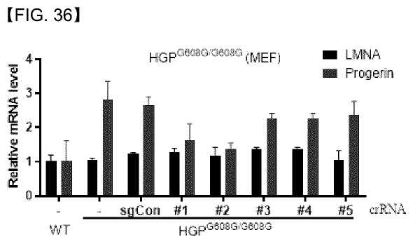

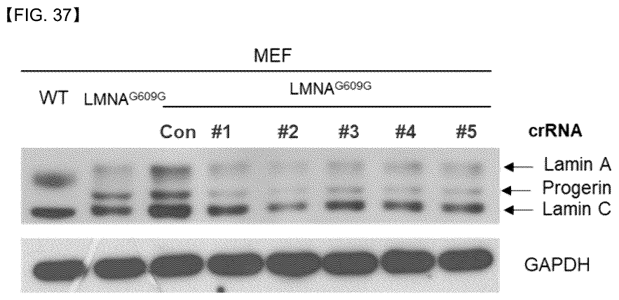

As a result, as shown in FIG. 36, progerin homozygote (Ho) mouse embryonic fibroblasts expressing progerin showed significantly lower expression of progerin compared with the experimental group into which the compositions were not introduced, due to the operation of the composition of the present disclosure (CRISPR-Cas13d #1, #2, SEQ ID NOs: 11 and 12). Like Experimental Example 5-1, sgRNA encoded by SEQ ID NO: 2 showed excellent progerin mRNA expression level reduction effects, and even when the expression of protein was checked again, similar results were obtained (FIG. 37).

### Experimental Example 6: Confirmation of the HGPS Phenotype Alleviation Effect of the Recombinant Expression Vector of Present Disclosure (In Vitro)

### Experimental Example 6-1. Confirmation of Nuclear Shape Recovery Effect

Nuclear shape abnormalities and recovery effects of the experimental groups prepared in Experimental Example 3-2 were confirmed.

Nuclear shape was confirmed through Immunocytochemistry (ICC) analysis method, and ICC method was verified through the following process. A group of mouse embryonic fibroblasts treated with the composition of the present disclosure (lentivirus RfxCas13d #2, SEQ ID NO: 12) and a control group of mouse embryonic fibroblasts not treated, were prepared, and then fixed using 10% formalin. Next, after treatment with 0.5% Triton X-100 for nuclear staining and blocking using 10% Normal Goat Serum, the groups were incubated at 4° C. with primary antibodies (Lamin A/C, Cat #4777S, Cell signaling technology) overnight. The next day, after sufficient washing, blocking was performed using 10% Normal Goat Serum again, and incubation was performed at room temperature for 1 hour using a secondary antibody to which Alexa Fluor 594 was bound. After final washing, the nuclei was stained with mounting medium, and then the coverslip was closed, and fluorescence thereof was observed using a confocal microscope. Incubation was performed for 1 hour at room temperature using a secondary antibody bound to peroxidase.

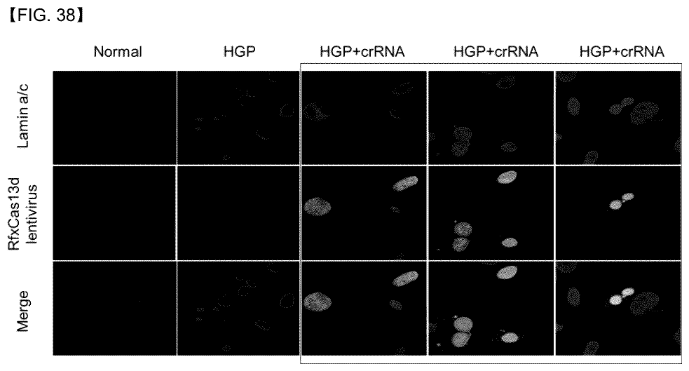

As a result, as shown in FIG. 38, the mouse embryonic fibroblast of the wild-type mouse had an oval nuclear shape regardless of whether or not the composition of present disclosure (CRISPR-Cas13a #2 lentivirus, SEQ ID NO: 12) was introduced. (Normal in FIG. 38). On the other hand, regarding the homozygote (Ho) mouse embryonic fibroblast experimental group (HGP in FIG. 38) to which the composition of the present disclosure (CRISPR-Cas13a #2 lentivirus, SEQ ID NO: 12) was not introduced, compared to the wild-type cell line, the abnormal shape (hypertrophy, distortion, splitting) could be confirmed. In addition, in the case of the experimental group (HGPS+crRNA in FIG. 38) into which the composition of the present disclosure (CRISPR-Cas13d #2 lentivirus, SEQ ID NO: 12) was introduced, the abnormal nuclear shape was recovered to such an extent that is close to an elliptical shape, compared to the experimental group into which the composition was not introduced.

### Experimental Example 6-2. Confirmation of the Effect of Reducing the Expression of Genes Related to Aging and Active Oxygen Species Induced by HGPS

The expression of aging-related genes (p21, p16, PAI-1, MMP1) and active oxygen-related genes (Catalase, Nrf2, SOD2) of the experimental groups prepared in Experimental Example 3-2 was confirmed.

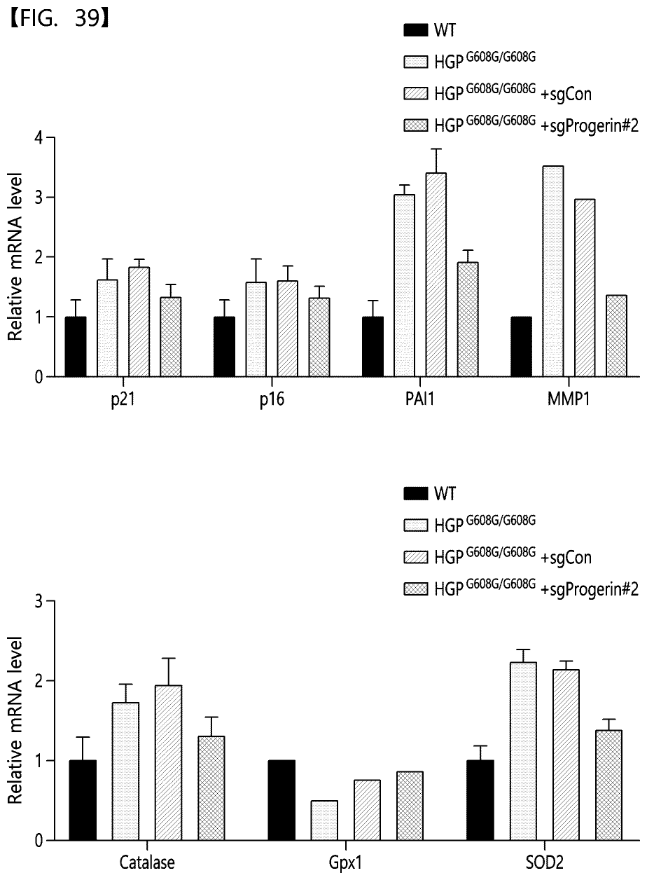

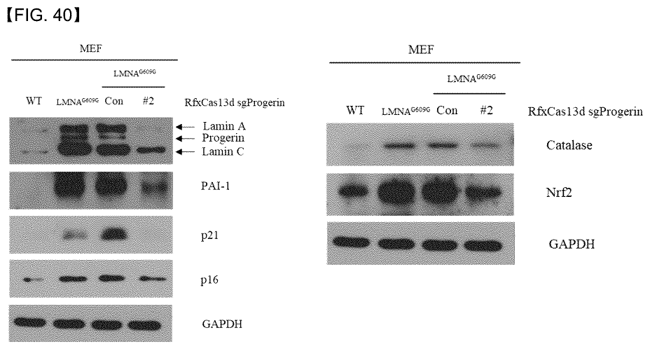

As a result, as confirmed from FIG. 39, in the case of the homozygote (Ho) (HO), the mouse embryonic fibroblast experimental group (LMNA^{G609G/G609G} of FIG. 39) into which the composition of the present disclosure (CRISPR-Cas13d #2 lentivirus, SEQ ID NO: 12) was not introduced, showed increased expressions of aging-related genes (p21, p16, PAI-1, MMP1) and active oxygen-related genes (Catalase, SOD2) compared to wild-type cell lines. In addition, it was confirmed that the experimental group (LMNA^{G609G/G609G} sgProgerin #2 in FIG. 39) into which the composition of the present disclosure (CRISPR-Cas13d #2 lentivirus, SEQ ID NO: 12) was introduced, showed reduced expressions of aging-related genes (p21, p16, PAI-1, MMP1) and active oxygen-related genes (Catalase, SOD2) compared to the experimental group into which the composition was not introduced. The results showed similar results when the expression of a protein was confirmed again (FIG. 40).

### Experimental Example 6-3. Confirmation of the Alleviating Effect on Aging Induced by HGPS

The aging effect on the experimental groups prepared in Experimental Example 3-2 was confirmed, and the effect through introduction of the composition of the present disclosure (CRISPR-Cas13d #2) was verified.

Confirmation of the senescence effect was verified using the β-gal staining method. For staining, Senescence β-Galactosidase Staining Kit (CST, #9860) was used. A group of mouse embryonic fibroblast cells treated with the composition of the present disclosure (lentivirus RfxCas13d #2, SEQ ID NO: 12) and a control group not treated with the composition, were treated with 1× fixing solution, exposed to room temperature for 10 minutes, and then washed with PBS. Then, a β-Galactosidase staining reagent was added thereto, followed by sealing. The resultant cells were stored for 2 days at 37° C. in an incubator without carbon dioxide. After storage, samples are taken out and stained using an optical microscope.

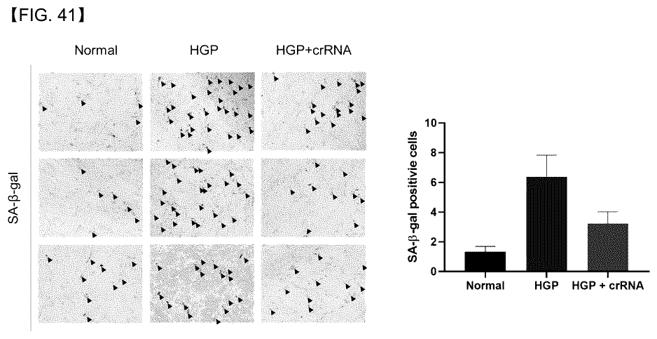

As a result, as confirmed in FIG. 41, in the homozygote (Ho), in the case of the mouse embryonic fibroblast experimental group (LMNA^{G609G/G609G} of FIG. 41) to which the composition of the present disclosure (CRISPR-Cas13d #2 lentivirus, SEQ ID NO: 12) was introduced, the green-stained portion (the portion indicated by the arrow) was increased compared to the wild-type cell line. Furthermore, it can be seen that in the case of the experimental group) (LMNA^{G609G/G609G} sgProgerin #2 in FIG. 41) to which the composition of the present disclosure (CRISPR-Cas13d #2 lentivirus, SEQ ID NO: 12) was introduced, the green-stained portion was reduced compared to the experimental group to which the composition was not introduced.

### Experimental Example 6-4. Confirmation of the Effect of Reducing the Expression of Progerin in HGPS Patient-Derived Fibroblasts

A control and fibroblasts of HGPS patients were purchased from the Coriell Institute and used for experiments to confirm the expression of progerin mRNA when treated with a recombinant expression vector according to the present disclosure.

Through Experimental Example 7-3, it was confirmed the effect of sgRNA encoded by SEQ ID NO: 2 (lentivirus RfxCas13d #2, confirmed through a recombinant expression vector of SEQ ID NO: 12) was the best, so lentivirus RfxCas13d #2 (SEQ ID NO: 12) was used to continue the experiment. The control and fibroblasts of progeria patients were treated with lentivirus RfxCas13d #2 (SEQ ID NO: 12), and then the effect of alleviating the expression of progerin was confirmed by RT-PCR.

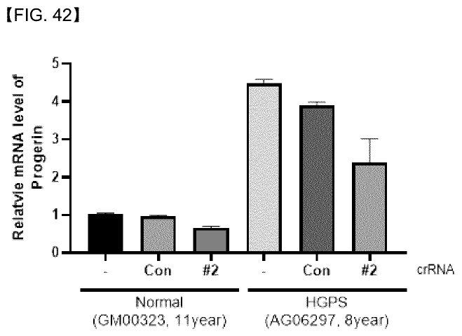

As a result, as confirmed in FIG. 42, it was confirmed that the expression of progerin was increased in the case of HGPS patient samples compared to the control. In the case of an experiment using HGPS patient samples, the experimental group, into which lentivirus RfxCas13d #2 (SEQ ID NO: 12) was introduced, showed a significantly lower expression of progerin compared to the experimental group into which the same was not introduced.

Through the above results, it was confirmed that, in the case of the sgRNA encoded by SEQ ID NO: 2, even patient-derived cells showed excellent progerin mRNA expression level reduction effects.

The present disclosure has been described above in terms of its preferred embodiments. Those skilled in the art to which the present disclosure pertains will be able to understand that the present disclosure can be implemented in a modified form without departing from the essential characteristics of the present disclosure. Therefore, the disclosed embodiments should be considered from an illustrative rather than a limiting point of view. The scope of the present disclosure is shown in the claims rather than the foregoing description, and all differences within the equivalent scope will be construed as being included in the present disclosure.

1. A polynucleotide encoding a gRNA that hybridizes to progerin mRNA, wherein the polynucleotide is one of SEQ ID NOs: 1 to 5.

2. The polynucleotide of claim 1, wherein the polynucleotide is a gRNA derived from the polynucleotide.

3. The polynucleotide of claim 2, wherein the gRNA hybridizes to genes encoding exon 11 and exon 12 of an LMNA gene.

4. (canceled)

5. A recombinant expression vector comprising a polynucleotide sequence of one of SEQ ID NOs: 1 to 5 encoding a gRNA that hybridizes to progerin mRNA or a sequence complementary thereto.

6. The recombinant expression vector of claim 5, wherein the recombinant expression vector further comprises a polynucleotide sequence encoding Cas13.

7. The recombinant expression vector of claim 5, wherein the recombinant expression vector consists of one of SEQ ID NOs: 6 to 15.

8. (canceled)

9. (canceled)

10. (canceled)

11. A method of treating progeria and alleviating natural aging, the method comprising administering a composition for inhibiting the expression of progerin, comprising the recombinant expression vector of claim 5 as an active ingredient to a subject in need thereof.

12. The method of claim 11, wherein the composition for inhibiting the expression of progerin further comprises Cas13 expression vector.

13. (canceled)


SEQUENCE LISTING (35 sequences)

SEQ ID NO: 1 - DNA, 28 bp, synthetic construct
  source 1..28: mol_type = other DNA
ctgggggctctgggctcctgagccgctg

SEQ ID NO: 2 - DNA, 28 bp, synthetic construct
  source 1..28: mol_type = other DNA
gttctgggggctctgggctcctgagccg

SEQ ID NO: 3 - DNA, 28 bp, synthetic construct
  source 1..28: mol_type = other DNA
cagttctgggggctctgggctcctgagc

SEQ ID NO: 4 - DNA, 28 bp, synthetic construct
  source 1..28: mol_type = other DNA
gcagttctgggggctctgggctcctgag

SEQ ID NO: 5 - DNA, 28 bp, synthetic construct
  source 1..28: mol_type = other DNA
gctgcagttctgggggctctgggctcct

SEQ ID NO: 6 - DNA, 11491 bp, synthetic construct
  source 1..11491: mol_type = other DNA
ggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttgcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgtctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaacaccgatttagactaccccaaaaacgaaggggactaaaacctgggggctctgggctcctgagccgctgtttttttgaattctgatgcggtattttctccttacgcatctgtgcggtatttcacaccgcatacgtcaaagcaaccatagtcctcgagtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgccccaggcggggcggggcggggcgaaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgcttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgggtgagccgccaccatggctggagctagccccaaaaagaagaggaaagtgggatccatgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacggtatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagttggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaaggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgcctaatgtacaaggaattctaactagagctcgcctgca

SEQ ID NO: 7 - DNA, 11491 bp, synthetic construct
  source 1..11491: mol_type = other DNA
ggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccacctccccgcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttgcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgtctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaacaccgatttagactaccccaaaaacgaaggggactaaaacgttctgggggctctgggctcctgagccgtttttttgaattctgatgcggtattttctccttacgcatctgtgcggtatttcacaccgcatacgtcaaagcaaccatagtcctcgagtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgggtgagccgccaccatggctggagctagccccaaaaagaagaggaaagtgggatccatgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacggtatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagttggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaaggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgcctaatgtacaaggaattctaactagagctcgcctgca

SEQ ID NO: 8 - DNA, 11491 bp, synthetic construct
  source 1..11491: mol_type = other DNA
ggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttgcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgtctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaacaccgatttagactaccccaaaaacgaaggggactaaaaccagttctgggggctctgggctcctgagctttttttgaattctgatgcggtattttctccttacgcatctgtgcggtatttcacaccgcatacgtcaaagcaaccatagtcctcgagtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgccccaggcggggcggggcggggcgaaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgcttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgggtgagccgccaccatggctggagctagccccaaaaagaagaggaaagtgggatccatgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacggtatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagttggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaaggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgcctaatgtacaaggaattctaactagagctcgcctgca

SEQ ID NO: 9 - DNA, 11491 bp, synthetic construct
  source 1..11491: mol_type = other DNA
ggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccacctccccgcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttgcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgtctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaacaccgatttagactaccccaaaaacgaaggggactaaaacgcagttctgggggctctgggctcctgagtttttttgaattctgatgcggtattttctccttacgcatctgtgcggtatttcacaccgcatacgtcaaagcaaccatagtcctcgagtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgggtgagccgccaccatggctggagctagccccaaaaagaagaggaaagtgggatccatgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacggtatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagttggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaaggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgcctaatgtacaaggaattctaactagagctcgcctgca

SEQ ID NO: 10 - DNA, 11491 bp, synthetic construct
  source 1..11491: mol_type = other DNA
ggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccacctccccgcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttgcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgtctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaacaccgatttagactaccccaaaaacgaaggggactaaaacgctgcagttctgggggctctgggctccttttttttgaattctgatgcggtattttctccttacgcatctgtgcggtatttcacaccgcatacgtcaaagcaaccatagtcctcgagtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgggtgagccgccaccatggctggagctagccccaaaaagaagaggaaagtgggatccatgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacggtatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagttggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaaggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgcctaatgtacaaggaattctaactagagctcgcctgca

SEQ ID NO: 11 - DNA, 12637 bp, synthetic construct
  source 1..12637: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacctgggggctctgggctcctgagccgctgtttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaataagcgacctgccgccacaaagaaggctggacaggctaagaagaagaaaggatccggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 12 - DNA, 12637 bp, synthetic construct
  source 1..12637: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgttctgggggctctgggctcctgagccgtttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaataagcgacctgccgccacaaagaaggctggacaggctaagaagaagaaaggatccggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 13 - DNA, 12637 bp, synthetic construct
  source 1..12637: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaaccagttctgggggctctgggctcctgagctttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaataagcgacctgccgccacaaagaaggctggacaggctaagaagaagaaaggatccggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 14 - DNA, 12637 bp, synthetic construct
  source 1..12637: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgcagttctgggggctctgggctcctgagtttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaataagcgacctgccgccacaaagaaggctggacaggctaagaagaagaaaggatccggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 15 - DNA, 12637 bp, synthetic construct
  source 1..12637: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgctgcagttctgggggctctgggctccttttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaataagcgacctgccgccacaaagaaggctggacaggctaagaagaagaaaggatccggaggaggtggaagcggaggaggaggaagcggaggaggaggtagcgtgagtaaaggtgaagaactcttcactggagtagtgcccattctggtagagcttgatggagatgtaaatggacataaattctccgtcaggggcgaaggcgaaggggacgccacgaatggtaagctgactctgaaattcatctgtacgacgggcaaactgcccgtcccatggcctacactcgtaacgaccctcacctacggcgtgcaatgcttttctcgatatcccgaccacatgaaacagcatgactttttcaagtctgcaatgcctgaaggttatgttcaagaaaggaccatcagctttaaggatgatggtacatataaaacccgagccgaggttaaatttgaaggggacactctggttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagggatccgagggagctcccaagaaaaagcgcaaggtaggtagttcctacccatacgatgttccagattacgcttatccctacgacgtgcctgattatgcatacccatatgatgtccccgactatgccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 16 - DNA, 23 bp, synthetic construct
  source 1..23: mol_type = other DNA
gtgagagttatctgaccgtaagg

SEQ ID NO: 17 - DNA, 23 bp, synthetic construct
  source 1..23: mol_type = other DNA
caaaataggttagccttgcgggg

SEQ ID NO: 18 - DNA, 20 bp, synthetic construct
  source 1..20: mol_type = other DNA
cccctatgtccacttcagga

SEQ ID NO: 19 - DNA, 19 bp, synthetic construct
  source 1..19: mol_type = other DNA
aggatcctctctggctcca

SEQ ID NO: 20 - DNA, 20 bp, synthetic construct
  source 1..20: mol_type = other DNA
cccctatgtccacttcagga

SEQ ID NO: 21 - DNA, 21 bp, synthetic construct
  source 1..21: mol_type = other DNA
tggtcatagctgtttcctgtg

SEQ ID NO: 22 - DNA, 20 bp, synthetic construct
  source 1..20: mol_type = other DNA
acttatgggaaccccttcca

SEQ ID NO: 23 - DNA, 20 bp, synthetic construct
  source 1..20: mol_type = other DNA
atttgttcagggctccacag

SEQ ID NO: 24 - DNA, 20 bp, synthetic construct
  source 1..20: mol_type = other DNA
acttatgggaaccccttcca

SEQ ID NO: 25 - DNA, 20 bp, synthetic construct
  source 1..20: mol_type = other DNA
gataaatctggagccggtga

SEQ ID NO: 26 - DNA, 20 bp, synthetic construct
  source 1..20: mol_type = other DNA
ccttgctgacttaccggttc

SEQ ID NO: 27 - DNA, 20 bp, synthetic construct
  source 1..20: mol_type = other DNA
acatgatgctgcagttctgg

SEQ ID NO: 28 - DNA, 20 bp, synthetic construct
  source 1..20: mol_type = other DNA
ccttgctgacttaccggttc

SEQ ID NO: 29 - DNA, 20 bp, synthetic construct
  source 1..20: mol_type = other DNA
acatgatgctgcagttctgg

SEQ ID NO: 30 - DNA, 18 bp, synthetic construct
  source 1..18: mol_type = other DNA
tctgcctccagtgtcacg

SEQ ID NO: 31 - DNA, 20 bp, synthetic construct
  source 1..20: mol_type = other DNA
acatgatgctgcagttctgg

SEQ ID NO: 32 - DNA, 18 bp, synthetic construct
  source 1..18: mol_type = other DNA
agcccagagcccccagaa

SEQ ID NO: 33 - DNA, 27 bp, synthetic construct
  source 1..27: mol_type = other DNA
caaagggttatttttctttggcttcaa

SEQ ID NO: 34 - DNA, 20 bp, synthetic construct
  source 1..20: mol_type = other DNA
tcaagaaggtggtgaagcag

SEQ ID NO: 35 - DNA, 20 bp, synthetic construct
  source 1..20: mol_type = other DNA
tcgctgttgaagtcagagga